SEQUENCE LISTING (13 sequences)

SEQ ID NO: 1 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 1366 aa, Homo sapiens [sequence omitted]

SEQ ID NO: 3 - protein, 1464 aa, Homo sapiens; identical to SEQ ID NO: 1

SEQ ID NO: 4 - protein, 1366 aa, Homo sapiens [sequence omitted]

SEQ ID NO: 5 - protein, 1489 aa, Homo sapiens
MAHARVLLLALAVLATAAVAVASSSSFADSNPIRPVTDRAASTLAQLQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPAGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKTSRLPIIDVAPLDVGAPDQEFGFDVGPVCFL

SEQ ID NO: 6 - protein, 1389 aa, Homo sapiens
MAHARVLLLALAVLATAAVAVASSSSFADSNPIRPVTDRAASTLAQLLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPTGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAHGAVGAPGPAGATGDRGEAGAAGPAGPAGPRGSPGERGEVGPAGPNGFAGPAGAAGQPGAKGERGGKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGFKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMEAIKVYCDFPTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADHEFFVDIGPVCFK

SEQ ID NO: 7 - DNA, 5927 bp, Homo sapiens
tcgtcggagcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaacataa

SEQ ID NO: 8 - DNA, 5411 bp, Homo sapiens
gtgtcccatagtgtttccaaacttggaaagggcgggggagggcgggaggatgcggagggcggaggtatgcagacaacgagtcagagtttccccttgaaagcctcaaaagtgtccacgtcctcaaaaagaatggaaccaatttaagaagccagccccgtggccacgtcccttcccccattcgctccctcctctgcgcccccgcaggctcctcccagctgtggctgcccgggcccccagccccagccctcccattggtggaggcccttttggaggcaccctagggccagggaaacttttgccgtataaatagggcagatccgggctttattattttagcaccacggcagcaggaggtttcggctaagttggaggtactggccacgactgcatgcccgcgcccgccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 9 - DNA, 4467 bp, Homo sapiens
atggctcacgctcgtgttctcctcctcgctctcgctgttttggcaacagctgctgtggctgtggcttctagttcttcttttgctgattcaaaccctattagacctgttactgatagagcagcttccactttggctcaattgcaagaggagggccaggttgagggccaagatgaggatatccctccaattacatgcgtgcaaaatggcttgcgttaccacgatagggatgtgtggaaacctgaaccttgtcgtatctgtgtgtgtgataacggcaaggtgctctgcgatgatgttatctgcgatgagacaaaaaattgccctggcgctgaagttcctgagggcgagtgttgccctgtgtgccctgatggttccgagtccccaactgatcaggaaactactggcgtggagggcccaaaaggagatactggtccacgtggtcctaggggtccagcaggtcctccaggtagagatggtattccaggccagcctggattgccaggaccaccaggcccacctggcccaccaggacctcctggtcttggtggaaatttcgctccacaactctcttatggctatgatgagaagtcaacaggtggtatttccgttccaggtcctatgggaccatccggaccaagaggtctcccaggtcctccaggtgctcctggacctcaaggctttcaaggacctccaggcgaaccaggagaaccaggcgcttctggaccaatgggcccaaggggaccacctggcccaccaggaaaaaatggcgatgatggcgaagctggaaagcctggtcgtcctggagagagaggtcctcctggcccacagggtgcaagaggcttgccaggaactgctggcttgcctggaatgaagggacataggggcttctccggcctcgatggcgctaagggtgatgctggccctgctggaccaaagggcgagccaggttcccctggagaaaacggtgctcctggacaaatgggtcctcgtggacttccaggagaaaggggtcgtccaggcgctccaggaccagcaggtgctaggggaaacgatggtgcaacaggcgctgctggccctcctggcccaactggtcctgctggccctccaggattcccaggcgcagttggagctaaaggagaagcaggaccacagggccctaggggttctgaaggacctcagggtgttagaggtgaaccaggtcctccaggcccagctggagcagctggtccagcaggaaatccaggtgctgatggtcaacctggagctaagggcgctaatggcgcaccaggtatcgcaggcgcaccaggttttcctggcgctagaggcccaagtggtcctcaaggaccaggtggaccaccaggtccaaaaggcaattctggcgaacctggcgctccaggttctaaaggagatactggtgctaaaggcgaaccaggacctgttggtgttcagggtcctcctggtcctgctggagaagaaggaaaaagaggtgctcgtggagaaccaggaccaactggacttcctggacctcctggtgaacgtggcggacctggctcaaggggtttccctggagctgatggagtggcaggtccaaaaggccctgctggagagagaggttcaccaggtccagctggtcctaagggctcccctggtgaagcaggtagaccaggcgaagcaggattgccaggcgcaaagggattgacaggctctcctggtagtcctggcccagatggaaaaacaggcccaccaggtccagcaggacaagatggacgtccaggcccaccaggtcctcctggagcaaggggacaagctggcgttatgggttttccaggacctaaaggtgctgctggagagccaggaaaggcaggtgaaagaggagttcctggtccaccaggagcagtgggtcctgctggcaaagatggtgaagctggagcacagggccctccaggccctgctggcccagctggcgaacgtggagaacaaggcccagctggtagtccaggatttcaaggattgcctggccctgctggccctccaggagaagcaggaaaacctggagaacaaggagttcctggtgatttgggagcacctggaccttcaggagcacgtggtgaaagaggcttccctggcgagaggggtgttcaaggtccaccaggtccagcaggacctagaggtgctaatggcgctcctggcaacgatggagcaaaaggtgatgctggtgctcctggcgcacctggaagtcagggtgctcctggattgcaaggaatgcctggagagaggggtgctgctggcttgccaggcccaaagggcgataggggtgatgctggaccaaaaggtgctgatggatccccaggaaaagatggagttcgtggtcttactggcccaatcggacctccaggccctgctggcgctccaggtgataagggcgaaagtggcccaagtggacctgctggacctactggtgctagaggtgcacctggtgataggggtgaacctggaccacctggtccagctggttttgctggtcctcctggagctgatggacaacctggcgcaaagggtgaaccaggtgatgctggcgcaaagggagatgctggtccacctggacctgctggtccagcaggcccccctgggccaatcggtaatgttggagcaccaggtgctaagggagctaggggttccgctggtccacctggagcaacaggatttccaggcgctgctggtagagttggcccaccaggcccatccggaaacgcaggccctcctggtcctccaggtcctgctggcaaggagggtggcaaaggaccaaggggcgaaactggccctgctggtagacctggcgaagttggccctcctggaccaccaggtccagcaggagaaaaaggttccccaggagctgatggcccagctggtgctccaggaactccaggccctcaaggtattgctggacagagaggcgttgtgggactccctggtcaaaggggagagagaggatttccaggcttgccaggacctagtggagaacctggaaaacaaggcccatcaggcgctagtggagagcgtggacctcctggccctatgggacctcctggattggctggcccacctggcgaatcaggtcgtgaaggcgcaccaggcgcagaaggatcacctggaagagatggatcccctggtgctaaaggcgatcgtggagaaactggtccagcaggcccaccaggcgcaccaggtgcacctggcgctccaggacctgtgggaccagctggaaaatccggagataggggcgagacaggcccagcaggaccagctggacctgttggccctgctggcgctcgtggaccagcaggacctcaaggaccaaggggagataagggagaaacaggcgaacaaggcgataggggcattaagggtcataggggttttagtggcctccagggtcctcctggcccacctggatcaccaggagaacagggaccatctggtgcttccggcccagctggtccaagaggacctccaggatcagctggtgcacctggaaaagatggtcttaacggtctcccaggaccaatcggccctccaggacctagaggaagaacaggagatgctggccctgttggccctccaggacctcctggtccaccaggtccacctggtcctccatcagctggattcgatttttcatttcttccacagccaccacaagagaaagctcacgatggcggcagatattaccgtgctgatgatgctaacgttgttagggatagagatttggaagtggatacaactttgaaatccctctcccagcaaattgaaaacattagatctccagaaggttcacgtaaaaacccagctagaacatgtcgtgatttgaaaatgtgtcactccgattggaaaagtggtgaatactggattgatccaaatcagggctgtaatctcgatgctatcaaagttttctgtaacatggaaacaggcgaaacatgcgtttatcctactcaaccttccgtggctcagaaaaattggtacatctcaaaaaatcctaaagataagaggcacgtttggttcggtgaaagtatgactgatggatttcaatttgagtacggcggtcaaggtagtgatccagctgatgtggctattcaactcacatttttgcgtcttatgtccacagaggcatcacaaaacatcacttaccactgcaaaaacagtgtggcttatatggatcaacaaacaggaaaccttaagaaggctcttcttttgaagggctcaaacgagattgagattagagcagagggcaactcaaggtttacttattcagttactgttgatggctgcacttcacatactggcgcttggggtaaaacagttatcgagtataagactacaaaaacatcaagactcccaatcattgatgttgctcctctcgatgttggcgctcctgatcaagagttcggttttgatgtgggcccagtttgtttcctc

SEQ ID NO: 10 - DNA, 4167 bp, Homo sapiens
atggctcacgctcgtgttctcctcctcgctctcgctgttttggcaacagctgctgtggctgtggcttcaagttctagttttgctgattccaacccaattcgtccagttactgatagagcagcttccactttggctcaattgcttcaagaagaaactgtgaggaagggccctgctggcgataggggccctaggggcgaaaggggtccaccaggacctccaggcagggatggcgaagatggtccaactggccctcctggacctcctggccctccagggccacccggcttgggcggaaacttcgcagctcaatacgatggcaagggtgttggtcttggtcctggtcctatgggcttgatgggacctagaggcccacctggtgctgctggtgctcctggaccacagggttttcagggaccagctggcgagccaggagagccaggccaaacaggaccagctggtgcaaggggacctgctggacctcctggaaaagctggtgaagatggtcacccaggcaaaccaggacgtcctggcgaaagaggtgttgttggaccacaaggcgctaggggatttccaggtacacctggattgccaggttttaagggcattcgtggtcataacggcctcgatggattgaagggacagcctggcgcacctggcgttaagggtgaacctggagcaccaggtgaaaacggtactcctggccagactggtgcaagaggactcccaggtgaaaggggtagagttggtgctcctggacctgctggagctaggggtagtgatggtagtgttggtcctgtgggccctgctggtccaatcggttccgctggcccacctggattcccaggcgctccaggacctaaaggagaaatcggtgctgtgggtaacgcaggtcctactggtccagcaggtcctcgtggagaagtgggattgccaggactttctggtccagtgggccctccaggcaaccctggagctaacggcttgacaggagctaaaggcgcagcaggactccctggagtggctggcgcaccaggattgcctggtccaaggggtatcccaggccctgttggcgcagctggagctactggtgcacgtggacttgttggcgaaccaggccctgctggatcaaaaggcgagtctggaaataagggagaacctggttctgctggacctcaaggtcctcctggaccttctggagaagaaggaaaaaggggaccaaatggcgaggctggatcagcaggtccaccaggaccacctggacttcgtggatcccctggtagtagaggacttccaggcgctgatggtagagcaggcgttatgggaccaccaggaagtagaggagcatccggtccagcaggagttaggggtcctaacggagatgctggtagaccaggtgaaccaggtcttatgggcccaaggggcctcccaggtagtccaggaaatatcggccctgctggaaaagaaggccctgttggacttccaggtattgatggacgtcctggccctattggcccagcaggtgcaagaggagaacctggcaatattggatttccaggaccaaagggtccaacaggcgatcctggaaaaaatggagataagggtcatgctggattggcaggcgcaaggggcgctcctggtccagatggaaacaacggcgcacagggtccacctggccctcagggtgttcaaggcggaaaaggcgaacaaggcccagctggaccaccaggctttcaaggcttgccaggaccaagtggtccagcaggtgaagttggcaagccaggcgagcgtggacttcatggcgagtttggactccctggaccagcaggaccaaggggtgaaagaggccctcctggagagagtggcgctgctggaccaacaggcccaatcggtagtagaggtcctagtggacctccaggcccagatggaaataagggtgaaccaggagttgtgggcgctgttggaacagctggtccttcaggaccatcaggactcccaggcgagagaggcgctgctggcattcctggaggaaaaggtgaaaaaggcgaacctggcctccgtggcgaaatcggaaatcctggacgtgatggtgctcgtggtgcacacggcgctgtgggcgctccaggccctgctggtgctactggtgatagaggagaggctggcgcagctggcccagcaggtcctgctggcccaaggggtagtcctggtgaaagaggcgaagttggacctgctggccctaacggctttgctggccctgctggagcagcaggtcaacctggcgctaaaggtgaaaggggcggaaagggcccaaaaggtgaaaatggcgttgtgggaccaactggtccagtgggcgcagctggacctgctggtccaaatggaccaccaggaccagcaggtagtagaggagatggtggacctccaggaatgacaggttttccaggtgctgctggtagaacaggacctcctggtcctagtggtatttctggtccaccaggaccaccaggtcctgctggaaaagaaggattgaggggtccacgtggtgatcaaggaccagtgggcagaactggtgaagttggcgcagtgggaccacctggttttgctggagaaaagggcccttctggagaggcaggaacagctggtcctcctggtacacctggacctcaaggacttttgggtgcacctggtattctcggattgccaggaagtaggggcgaacgtggacttcctggcgtggcaggagcagttggagaacctggccctctcggaatcgcaggcccaccaggcgcaagaggaccaccaggagctgttggatcaccaggcgtgaatggtgcacctggcgaggctggtcgtgatggaaacccaggaaatgatggcccaccaggaagagatggtcaacctggacacaaaggcgagaggggctacccaggaaatattggcccagttggtgctgctggcgcaccaggcccacacggtccagttggaccagcaggaaaacacggtaatcgtggcgaaacaggcccttcaggcccagtgggacctgctggtgctgttggcccaagaggaccatctggacctcaaggcattagaggcgataagggagagcctggcgaaaaaggacctagaggcttgcctggttttaaaggacacaacggtctccaaggacttccaggtatcgctggtcatcatggagatcagggtgctcctggatcagtgggtccagcaggtcctagaggcccagcaggcccttccggtccagcaggaaaggatggacgtactggccaccctggaactgtgggccctgctggaattagaggtcctcaaggtcatcagggccctgctggccctccaggtccaccaggtcctccaggcccaccaggagtttcaggtggtggttacgattttggttacgatggtgatttttaccgtgctgatcaacctagaagtgctccttctctccgtcctaaagattatgaagttgatgctactttgaaatcacttaacaaccagattgagactcttctcacacctgagggatcaagaaagaatccagcacgtacatgccgtgatctcagacttagtcacccagagtggtcaagtggctattattggattgatcctaatcagggttgtacaatggaggctatcaaagtttactgtgattttccaactggagagacatgtattagggcacaacctgagaacattccagctaaaaattggtatcgttcctctaaagataagaaacatgtttggctcggagagactattaacgctggttctcagttcgagtataatgttgagggcgttacttctaaagagatggcaactcagctcgcttttatgagattgctcgctaactacgcatcccaaaacatcacttatcactgcaaaaattccattgcatatatggatgaggagacaggaaatttgaagaaagcagttattctccaaggtagtaacgatgttgagcttgtggctgagggaaatagtagattcacttacacagttttggtggatggatgctcaaagaaaactaatgagtggggcaagacaatcattgagtacaagacaaataagccttctaggctcccatttctcgatattgcacctcttgatatcggaggagctgatcacgagttttttgttgatatcggacctgtttgttttaag

SEQ ID NO: 11 - DNA, 1633 bp, Artificial sequence
  Synthetic sequence containing the coding regions of the vascular signal sequence of barley gene for Thiol protease aleurain precursor fused to the human Prolyl 4-hydroxylase beta subunit and flanking regions
ctcgagtaaaccatggctcatgctagggttttgcttttggctcttgctgttcttgctactgctgctgttgctgtggcttcttcttcatctttcgctgattctaacccaattaggccagtgactgatagagctgcttctactcttgctcaattggtcgacatggatgctccagaagaggaggatcacgttcttgtgcttaggaagtctaacttcgctgaagctcttgctgctcacaagtaccttcttgtggagttttatgctccttggtgcggacattgcaaagctcttgctccagagtatgctaaggctgctggaaagttgaaggctgagggatctgaaattaggcttgctaaagtggatgctactgaggagtctgatcttgctcaacagtacggagttaggggatacccaactattaagttcttcaggaacggagatactgcttctccaaaggagtatactgctggaagggaggctgatgatattgtgaactggcttaagaagagaactggaccagctgctactactcttccagatggagctgctgctgaatctcttgtggagtcatctgaggtggcagtgattggattcttcaaggatgtggagtctgattctgctaagcagttccttcaagctgctgaggctattgatgatattccattcggaattacttctaactctgatgtgttctctaagtaccagcttgataaggatggagtggtgcttttcaagaaattcgatgagggaaggaacaatttcgagggagaggtgacaaaggagaaccttcttgatttcattaagcacaaccagcttccacttgtgattgagttcactgagcagactgctccaaagattttcggaggagagattaagactcacattcttcttttccttccaaagtctgtgtctgattacgatggaaagttgtctaacttcaagactgctgctgagtctttcaagggaaagattcttttcattttcattgattctgatcacactgataaccagaggattcttgagttcttcggacttaagaaggaagagtgcccagctgttaggcttattactcttgaggaggagatgactaagtacaagccagagtctgaagaacttactgctgagaggattactgagttctgccacagattccttgagggaaagattaagccacaccttatgtctcaagagcttccagaggattgggataagcagccagttaaggtgttggtgggtaaaaacttcgaggatgtggctttcgatgagaagaagaacgtgttcgtggagttctacgcaccttggtgtggtcactgtaagcagcttgctccaatttgggataagttgggagagacttacaaggatcacgagaacattgtgattgctaagatggattctactgctaacgaggtggaggctgttaaggttcactctttcccaactttgaagttcttcccagcttctgctgataggactgtgattgattacaacggagaaaggactcttgatggattcaagaagttccttgagtctggaggacaagatggagctggagatgatgatgatcttgaggatttggaagaagctgaggagccagatatggaggaggatgatgatcagaaggctgtgtgatgagctc

SEQ ID NO: 12 - DNA, 1723 bp, Artificial sequence
  Synthetic sequence containing the coding regions of the vascular signal sequence of barley gene for Thiol protease aleurain precursor fused to the human Prolyl 4-hydroxylase alpha-1 subunit and flanking regions
ctcgagtaaaccatggctcatgctagggttttgcttttggctcttgctgttcttgctactgctgctgttgctgtggcttcttcttcatctttcgctgattctaacccaattaggccagtgactgatagagctgcttctactcttgctcaattggtcgacatgcacccaggattcttcacttctattggacagatgactgatcttattcacactgagaaggatcttgtgacttctcttaaggattacattaaggctgaggaggataagttggagcagattaagaagtgggctgagaagttggataggcttacttctactgctacaaaagatccagagggattcgttggtcatccagtgaacgctttcaagttgatgaagaggcttaacactgagtggagtgagcttgagaaccttgtgcttaaggatatgtctgatggattcatttctaaccttactattcagaggcagtacttcccaaatgatgaggatcaagtgggagctgctaaggctcttcttaggcttcaggatacttacaaccttgatactgatacaatttctaagggaaaccttccaggagttaagcacaagtctttccttactgctgaggattgcttcgagcttggaaaggttgcatacactgaggctgattactaccacactgagctttggatggaacaagctcttaggcaacttgatgagggagagatttctactattgataaggtgtcagtgcttgattacctttcttacgctgtgtaccagcagggtgatcttgataaggctcttttgcttactaagaagttgcttgagcttgatccagaacatcagagggctaacggaaaccttaagtacttcgagtacattatggctaaggaaaaggatgtgaacaagtctgcttctgatgatcagtctgatcaaaagactactccaaagaagaagggagtggctgttgattatcttcctgagaggcagaagtatgagatgttgtgtaggggagagggtattaagatgactccaaggaggcagaagaagttgttctgcaggtatcacgatggaaacaggaacccaaagttcattcttgctccagctaagcaagaagatgagtgggataagccaaggattattaggttccacgatattatttctgatgctgagattgagattgtgaaggatcttgctaagccaagacttaggagggctactatttctaaccctattactggtgatcttgagactgtgcactacaggatttctaagtctgcttggctttctggatacgagaacccagtggtgtctaggattaacatgaggattcaggatcttactggacttgatgtgtctactgctgaggagcttcaagttgctaactacggagttggaggacaatatgagccacacttcgatttcgctaggaaggatgagccagatgcttttaaggagcttggaactggaaacaggattgctacttggcttttctacatgtctgatgtttctgctggaggagctactgttttcccagaagtgggagcttctgtttggccaaagaagggaactgctgtgttctggtacaaccttttcgcttctggagagggagattactctactaggcatgctgcttgcccagttcttgttggaaacaagtgggtgtcaaacaagtggcttcatgagaggggacaagagtttagaaggccatgcactctttctgagcttgagtgatgagctc

SEQ ID NO: 13 - DNA, 2888 bp, Artificial sequence
  Synthetic sequence containing the coding regions of the vascular signal sequence of barley gene for Thiol protease aleurain precursor fused to the human Lysyl hydroxylase 3 and flanking regions
gcgaattcgctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattattctctgcttttgctctctatataaagagcttgtattttcatttgaaggcagaggcgaacacacacacagaacctccctgcttacaaaccagatcttaaaccatggctcacgctagggttttgcttcttgctcttgctgttcttgctactgctgctgttgctgtggcttcttcaagttctttcgctgattctaacccaattaggccagtgactgatagagctgcttctactcttgctcaattgagatctatgtctgatagaccaaggggaagggatccagttaatccagagaagttgcttgtgattactgtggctactgctgagactgaaggataccttagattccttaggagtgctgagttcttcaactacactgtgaggactcttggacttggagaagaatggaggggaggagatgttgctagaactgttggaggaggacagaaagtgagatggcttaagaaagagatggagaagtacgctgatagggaggatatgattattatgttcgtggattcttacgatgtgattcttgctggatctccaactgagcttttgaagaaattcgttcagtctggatctaggcttcttttctctgctgagtctttttgttggccagaatggggacttgctgagcaatatccagaagtgggaactggaaagagattccttaactctggaggattcattggattcgctactactattcaccagattgtgaggcagtggaagtacaaggatgacgatgatgatcagcttttctacactaggctttaccttgatccaggacttagggagaagttgtctcttaaccttgatcacaagtctaggattttccagaaccttaacggtgctcttgatgaggttgtgcttaagttcgataggaacagagtgaggattaggaacgtggcttacgatactcttcctattgtggtgcatggaaacggaccaacaaaactccagcttaactaccttggaaactacgttccaaacggatggactccagaaggaggatgtggattctgcaatcaggataggagaactcttccaggaggacaaccaccaccaagagttttccttgctgtgttcgttgaacagccaactccattccttccaagattccttcagaggcttcttcttttggattacccaccagatagggtgacacttttccttcacaacaacgaggttttccacgagccacacattgctgattcttggccacagcttcaggatcatttctctgctgtgaagttggttggtccagaagaagctctttctccaggagaagctagggatatggctatggatttgtgcaggcaggatccagagtgcgagttctacttctctcttgatgctgatgctgtgcttactaaccttcagactcttaggattcttattgaggagaacaggaaagtgattgctccaatgctttctaggcacggaaagttgtggtctaatttctggggtgctctttctcctgatgagtactacgctagatcagaggactacgtggagcttgttcagagaaagagagtgggagtttggaacgttccttatatttctcaggcttacgtgattaggggagatactcttaggatggagcttccacagagggatgttttctctggatctgatactgatccagatatggctttctgcaagtctttcagggataagggaattttccttcacctttctaaccagcatgagttcggaagattgcttgctacttcaagatacgatactgagcaccttcatcctgatctttggcagattttcgataacccagtggattggaaggagcagtacattcacgagaactactctagggctcttgaaggagaaggaattgtggagcaaccatgcccagatgtttactggttcccacttctttctgagcaaatgtgcgatgagcttgttgctgagatggagcattacggacaatggagtggaggtagacatgaggattctaggcttgctggaggatacgagaacgttccaactgtggatattcacatgaagcaagtgggatacgaggatcaatggcttcagcttcttaggacttatgtgggaccaatgactgagtctcttttcccaggataccacactaaggctagggctgttatgaacttcgttgtgaggtatcgtccagatgagcaaccatctcttaggccacaccacgattcttctactttcactcttaacgtggctcttaaccacaagggacttgattatgagggaggaggatgccgtttccttagatacgattgcgtgatttcttcaccaagaaagggatgggctcttcttcatccaggaaggcttactcattaccacgagggacttccaactacttggggaactagatatattatggtgtctttcgtggatccatgactgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccagaattcgcg

(19) United States
(12) Patent Application Publication
(10) Pub. No.: US 2012/0273993 A1
(43) Pub. Date: Nov. 1, 2012

(54) METHOD OF GENERATING COLLAGEN FIBERS

(75) Inventors: Oded Shoseyov, Karmei Yosef (IL); Amit Yaari, Kibbutz Ein Dror (IL)

(73) Assignee: CollPlant Ltd., Nes Ziona (IL)

(21) Appl. No.: 13/511,686

(22) PCT Filed: Nov. 24, 2010
(86) PCT No.: PCT/IL10/00984

§ 371 Date: May 24, 2012

Related U.S. Application Data
(60) Provisional application No. 61/263,839, filed on Nov. 24, 2009.

(51) Int. Cl. D01F 4/00; C07K 14/78

(52) U.S. Cl. 264/202; 530/356

(57) ABSTRACT

A method of generating a collagen fiber is disclosed. The method comprises extruding a solution of liquid crystalline collagen into a coagulating solution, thereby generating the collagen fiber. Fibers generated thereby are also disclosed as well as scaffolds comprising such fibers.

### FIELD AND BACKGROUND OF THE INVENTION

The present invention, in some embodiments thereof, relates to a method of generating collagen fibers.

Collagen is the principal structural protein in the body and constitutes approximately one-third of the total body protein. It comprises most of the organic matter of the skin, tendons, bones and teeth and occurs as fibrous inclusions in most other body structures. Some of the properties of collagen are its high tensile strength; its ion exchanging ability, due in part to the binding of electrolytes, metabolites and drugs; its low antigenicity, due to masking of potential antigenic determinants by the helical structure, and its low extensibility, semipermeability, and solubility. Furthermore collagen is a natural substance for cell adhesion. These properties make this protein suitable for fabrication of bioremodelable research products and medical devices such as implantable prostheses, cell growth substrates, and cellular and acellular tissue constructs.

Naturally, collagen is secreted by cells as a long triple-helical monomer, which polymerizes spontaneously into fibrils and strands, which often have a preferential orientation essential to the function of tissues such as skin, bone and nerve.

The exact structure of the collagen fibril is still unknown, but increasingly detailed models are becoming available, emphasizing the relation between fibril structure and function. Current models hint at a semi-crystalline (liquid crystal like) structure, combining a highly ordered arrangement in the axial direction and a short-range liquid-like order in the lateral direction.

Collagen in its monomeric form is soluble in cold acidic pH (˜pH 2) solutions, and can be precipitated in the form of fibrils by neutralizing the pH, increasing the temperature and/or the ionic strength. Fibrillogenesis is entropy driven—the loss of water molecules from monomer surfaces drives the collagen monomers out of solution and into assemblies with a circular cross-section, so as to minimize surface area.

The fibrils formed in-vitro display D-banding pattern of 67 nm wide cross striations typical of natural collagen fibrils formed in-vivo, but lack altogether the macroscopic order that is the basis of structural tissues. Fibrils precipitated out of bulk solutions form an entangled mesh reminiscent of spaghetti and not the neatly ordered arrays of fibrils observed in nature.

Collagen can be deposited from solution by a variety of processes including casting, lyophilization, electrospinning and other processes well known to one skilled in the art. In most of these procedures, collagen fibers of widely varying diameters and lengths from the micrometer range typical of conventional fibers down to the nanometer range are formed. Owing to their small diameters, electrospun fibers possess very high surface-to-area ratios and are expected to display morphologies and material properties very different from their conventional counterparts occurring in nature. Numerous attempts to direct or align collagen fibrils for manufacturing of collagen matrices have been performed, employing various methods. Major efforts are aimed at creating 2D (collagen surface) or 3D (collagen scaffold) matrices. Exemplary methods include: alignment by surface templating, chemical patterning, nanolithography, electrochemical fabrication, use of a magnetic field and by shear flow.

In vitro, collagen displays mesophase (liquid crystalline) properties at concentrations above ˜20 mg/ml (depending on acid concentration of the solvent). At concentrations between ˜20 to 50 mg/ml diffuse nematic phases appear in the bulk isotropic solution, observed as birefringent flakes. When the collagen concentration is increased, precholesteric patterns form—observed as spherulites, bands, or zigzag extinction patterns. Further increase in the concentration leads to formation of cholesteric patterns that become more and more compact until the entire sample displays characteristic fingerprint pattern.

At concentrations above 150 mg/ml, collagen fibrillar aggregates start to appear even in acidic solution, displaying the 67 nm banding typical of collagen fibrils, in a process reminiscent of a cholesteric-to-smectic (N*/SmA) transition.

U.S. Pat. No. 7,057,023 teaches spinning of liquid crystalline silk to generate silk fibers.

U.S. Patent Application No. 20070187862 teaches spinning a solution of liquid crystalline silk, wherein the solution is devoid of organic solvents to generate silk fibers.

U.S. Patent Application No. 20090069893 teaches formation of oriented collagen based materials from mesophase collagen by application of a shear force.

### SUMMARY OF THE INVENTION

According to an aspect of some embodiments of the present invention there is provided a method of generating a collagen fiber, the method comprising extruding a solution of liquid crystalline collagen into a coagulating solution, thereby generating the collagen fiber.

According to an aspect of some embodiments of the present invention there is provided a collagen fiber produced by the method of the present invention.

According to an aspect of some embodiments of the present invention there is provided a scaffold comprising the collagen fibers of the present invention.

According to an aspect of some embodiments of the present invention there is provided a method of generating a collagen matrix, the method comprising:

(a) extruding a solution of liquid crystalline collagen into a coagulating solution, thereby generating a collagen fiber; and

(b) casting the matrix from said collagen fiber, thereby generating the collagen matrix.

According to some embodiments of the invention, the method further comprises isolating the collagen fiber following said extruding.

According to some embodiments of the invention, the method further comprises drying the collagen fiber following said isolating.

According to some embodiments of the invention, the liquid crystalline collagen comprises collagen monomers.

According to some embodiments of the invention, the method further comprises polymerizing said collagen monomers following said extruding.

According to some embodiments of the invention, the method further comprises crosslinking said collagen monomers following said extruding.

According to some embodiments of the invention, the extruding is effected using a spinneret.

According to some embodiments of the invention, the coagulating solution further comprises a surfactant.

According to some embodiments of the invention, the extruding is effected by passing through an orifice comprising an inner diameter of 30 ga.

According to some embodiments of the invention, the coagulating solution comprises an organic solvent.

According to some embodiments of the invention, the solution of crystalline collagen monomers comprises hyaluronic acid (HA).

According to some embodiments of the invention, the solution of crystalline collagen monomers comprises a crosslinker.

According to some embodiments of the invention, the at least one organic solvent is selected from the group consisting of acetone, ethanol and isopropanol.

According to some embodiments of the invention, the collagen monomers comprise recombinant collagen monomers.

According to some embodiments of the invention, the collagen monomers comprise animal-derived collagen monomers.

According to some embodiments of the invention, the collagen monomers are present at a concentration of about 100 mg/ml in said solution of liquid crystalline collagen monomers.

According to some embodiments of the invention, the liquid solution of crystalline collagen monomers is an acidic solution.

According to some embodiments of the invention, the collagen fiber comprises an extinction pattern as displayed in FIGS. 1A-B.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C are images of mesophase collagen extruded fibers using a polarized light microscope. FIG. 1A—Ethanol as a coagulating solution. FIG. 1B—Acetone as a coagulating solution. FIG. 1C—A polarized light microscope image of reconstituted collagen fibers formed by extrusion of 1% collagen solution to fibrillogenesis buffer and dried in isopropanol [Kato et al., J Bone Joint Surg Am. 1991; 73:561-574].

FIGS. 2A-F are electron micrographs of collagen. FIG. 1A: Electron micrograph of a tenocyte located in a normal supraspinatus tendon. The nucleus (n) and cytoplasm (cy) have been labeled. The extracellular matrix shows collagen fibrils (c) in a variety of orientations. Uranyl acetate and lead citrate [Cell and matrix changes associated with pathological calcification of the human rotator cuff tendons. R. S. Archer, J. I. L. Bayley, C. W. Archer And S. Y. Ali. J. Anat. (1993). 182, pp. 1-12]. Bar-2 micron. FIG. 2B: Electron micrograph of a mesophase collagen injected into an isopropanol bath. Bar-2 micron. FIG. 2C: Electron micrograph of a mesophase collagen injected into fibrillogenesis buffer with 40% PEG bath. Bar-2 micron. FIG. 2D: TEM micrograph of pepsin soluble, reconstituted dilute collagen fibers. Bar-2 micron [Zeugolis et al., Applied Biomaterials. 86A(4), pp. 892-904. 1549-3296]. FIG. 2E: Thin section bullfrog tendon of adjacent fibers in the compression region of bullfrog tendon, showing the convolution and “kinking of their composing fibrils. Each bundle has a marked delimitation of its surface by an almost indistinguishable material (arrowheads). The existence of spaces between fibers can also be seen. 11000×. Bar=1 micron [Hernandes Faustino de Carvalho et al., Biol Cell (1994) 82, 59-65]. FIG. 2F: Electron micrograph of a dilute collagen injected into fibrillogenesis buffer with 10% PEG bath. Bar-1 micron [Caves et al., J Biomed Mater Res B Appl Biomater. 2010 April; 93(1):24-38].

FIGS. 3A-D are electron micrograph images of mesophase collagen extruded into acetone.

FIGS. 4A-D are electron micrograph images of mesophase collagen extruded into isopropanol.

FIGS. 5A-D are electron micrograph images of mesophase collagen extruded into ethanol.

FIGS. 6A-D are electron micrograph images of mesophase collagen extruded into high osmolarity buffer.

FIGS. 7A-D are electron micrograph images of mesophase collagen extruded into high osmolarity buffer and subsequent incubation in PBS.

FIGS. 8A-B are electron micrograph images of mesophase collagen extruded into high ionic strength buffer.

FIGS. 9A-D are electron micrograph images of mesophase collagen extruded into high ionic strength buffer and subsequent incubation in buffer 3.

### DESCRIPTION OF SPECIFIC EMBODIMENTS OF THE INVENTION

The present invention, in some embodiments thereof, relates to a method for generating collagen fibers.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Collagen matrix in many biological systems has a very highly ordered liquid crystal structure (mesophase). It is this natural state which provides collagen with its long-range orientation.

The highly ordered mesophase state of naturally occurring collagen can be mimicked in vitro by increasing the concentration of a solution of monomeric collagen above ˜20 mg/ml (depending on acid concentration of the solvent).

The present inventors propose that preservation of the crystalline order instilled by the mesophase state of collagen following extrusion, would allow for the generation of collagen fibers with a highly organized collagen structure, thereby providing the fiber with superior mechanical properties.

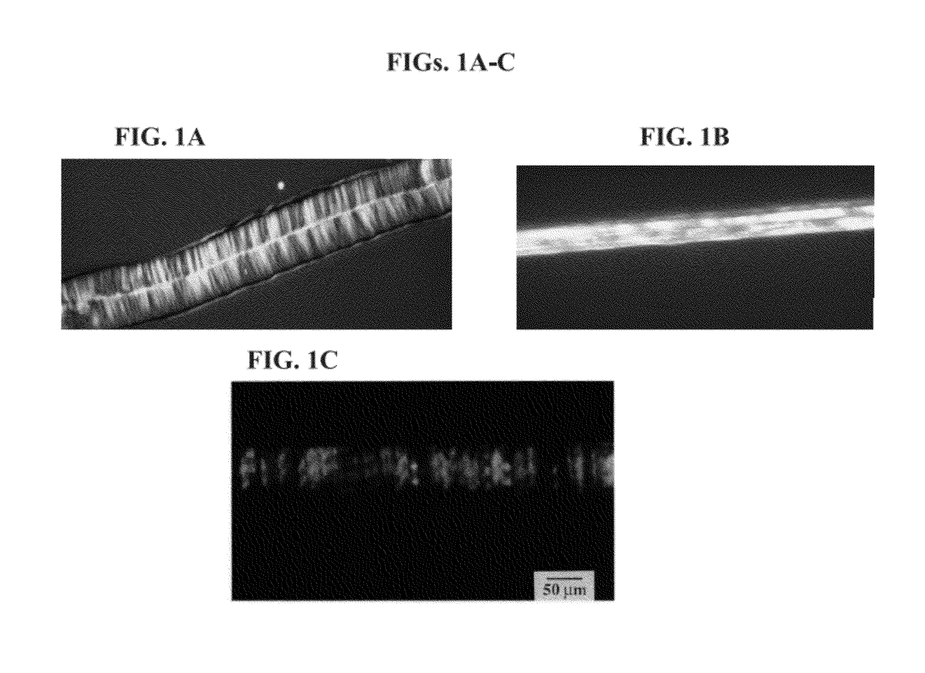

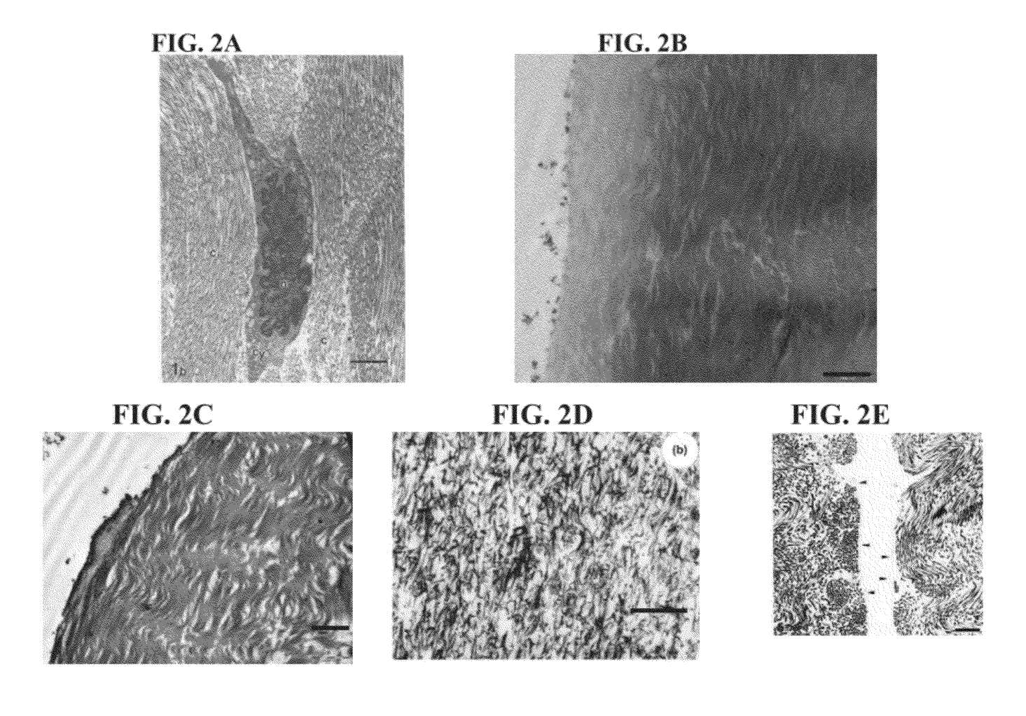

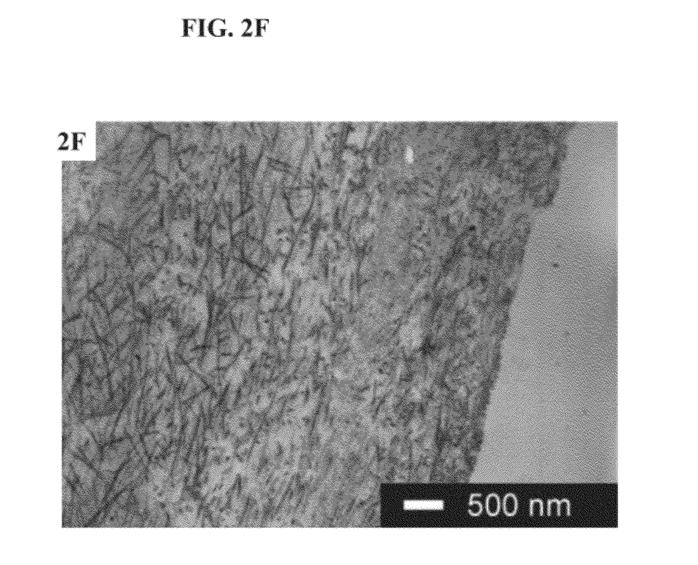

The present inventors showed that extruding fibers from mesophase collagen directly into a coagulating solution maintains and preserves the crystalline structure assumed by the collagen in the mesophase (FIGS. 2-9). For example, the present inventors showed that collagen mesophase extruded into isopropanol displayed a much stronger extinction pattern compared to collagen fibers which were not extruded from mesophase collagen (FIGS. 1A-C). The stronger extinction pattern was attributed to the higher degree of intrafibrillar order present in the mesophase extruded fibers relative to the soluble collagen extruded fibers.

Thus, according to one aspect of the present invention there is provided a method of generating a collagen fiber, the method comprising extruding a solution of liquid crystalline collagen into a coagulating solution, thereby generating the collagen fiber.

The term “collagen” as used herein, refers to a polypeptide having a triple helix structure and containing a repeating Gly-X-Y triplet, where X and Y can be any amino acid but are frequently the imino acids proline and hydroxyproline. According to one embodiment, the collagen is a type I, II, III, V, XI, or biologically active fragments therefrom.

A collagen of the present invention also refers to homologs (e.g., polypeptides which are at least 50%, at least 55%, at least 60%, at least 65%, at least 70%, at least 75%, at least 80%, at least 85%, at least 87%, at least 89%, at least 91%, at least 93%, at least 95% or more say 100% homologous to collagen sequences listed in Table 1 as determined using BlastP software of the National Center of Biotechnology Information (NCBI) using default parameters). The homolog may also refer to a deletion, insertion, or substitution variant, including an amino acid substitution, thereof and biologically active polypeptide fragments thereof.

Table 1 below lists examples of collagen NCBI sequence numbers.

TABLE 1

| Exemplary collagen NCBI sequence number | SEQ ID NO: |
|---|---|
| P02452 | 1 |
| P08123 | 2 |

According to one embodiment, the collagen of the present invention comprises a sufficient portion of its telopeptides such that under suitable conditions it is capable of forming fibrils.

Thus, for example, the collagen may be atelocollagen, a telocollagen or procollagen.

As used herein, the term “atelocollagen” refers to collagen molecules lacking both the N- and C-terminal propeptides typically comprised in procollagen and at least a portion of its telopeptides, but including a sufficient portion of its telopeptides such that under suitable conditions it is capable of forming fibrils.

The term “procollagen” as used herein, refers to a collagen molecule (e.g. human) that comprises either an N-terminal propeptide, a C-terminal propeptide or both. Exemplary human procollagen amino acid sequences are set forth by SEQ ID NOs: 3, 4, 5 and 6.

The term “telocollagen” as used herein, refers to collagen molecules that lack both the N- and C-terminal propeptides typically comprised in procollagen but still contain the telopeptides. The telopeptides of fibrillar collagen are the remnants of the N- and C-terminal propeptides following digestion with native N/C proteinases.

According to another embodiment, the collagen is devoid of its telopeptides and is not capable of undergoing fibrillogenesis.

According to another embodiment, the collagen is a mixture of the types of collagen above.

The collagen may be isolated from an animal (e.g. bovine, pig or human) or may be genetically engineered using recombinant DNA technology.

Methods of isolating collagen from animals are known in the art. Dispersal and solubilization of native animal collagen can be achieved using various proteolytic enzymes (such as porcine mucosal pepsin, bromelain, chymopapain, chymotrypsin, collagenase, ficin, papain, peptidase, proteinase A, proteinase K, trypsin, microbial proteases, and, similar enzymes or combinations of such enzymes) which disrupt the intermolecular bonds and remove the immunogenic non-helical telopeptides without affecting the basic, rigid triple-helical structure which imparts the desired characteristics of collagen (see U.S. Pat. Nos. 3,934,852; 3,121,049; 3,131,130; 3,314,861; 3,530,037; 3,949,073; 4,233,360 and 4,488,911 for general methods for preparing purified soluble collagen). The resulting soluble collagen can be subsequently purified by repeated precipitation at low pH and high ionic strength, followed by washing and re-solublization at low pH.

Plants expressing collagen chains and procollagen are known in the art, see for example, WO06035442A3; Merle et al., FEBS Lett. 2002 Mar. 27; 515(1-3):114-8. PMID: 11943205; and Ruggiero et al., 2000, FEBS Lett. 2000 Mar. 3; 469(1):132-6. PMID: 10708770; and U.S. Pat. Applications 2002/098578 and 2002/0142391 as well as U.S. Pat. No. 6,617,431 each of which are incorporated herein by reference.

It will be appreciated that the present invention also contemplates genetically modified forms of collagen/atelocollagen—for example collagenase-resistant collagens and the like [Wu et al., Proc Natl. Acad Sci, Vol. 87, p. 5888-5892, 1990].

Recombinant procollagen or telocollagen may be expressed in any non-animal cell, including but not limited to plant cells and other eukaryotic cells such as yeast and fungus.

Plants in which the human procollagen or telocollagen may be produced (i.e. expressed) may be of lower (e.g. moss and algae) or higher (vascular) plant species, including tissues or isolated cells and extracts thereof (e.g. cell suspensions). Preferred plants are those which are capable of accumulating large amounts of collagen chains, collagen and/or the processing enzymes described herein below. Such plants may also be selected according to their resistance to stress conditions and the ease at which expressed components or assembled collagen can be extracted. Examples of plants in which human procollagen may be expressed include, but are not limited to tobacco, maize, alfalfa, rice, potato, soybean, tomato, wheat, barley, canola, carrot, lettuce and cotton.

Production of recombinant procollagen is typically effected by stable or transient transformation with an exogenous polynucleotide sequence encoding human procollagen.

Exemplary polynucleotide sequences encoding human procollagen are set forth by SEQ ID NOs: 7, 8, 9 and 10.

Production of human telocollagen is typically effected by stable or transient transformation with an exogenous polynucleotide sequence encoding human procollagen and at least one exogenous polynucleotide sequence encoding the relevant protease.

The stability of the triple-helical structure of collagen requires the hydroxylation of prolines by the enzyme prolyl-4-hydroxylase (P4H) to form residues of hydroxyproline within the collagen chain. Although plants are capable of synthesizing hydroxyproline-containing proteins, the prolyl hydroxylase that is responsible for synthesis of hydroxyproline in plant cells exhibits relatively loose substrate sequence specificity as compared with mammalian P4H. Thus, production of collagen containing hydroxyproline only in the Y position of Gly-X-Y triplets requires co-expression of collagen and human or mammalian P4H genes [Olsen et al, Adv Drug Deliv Rev. 2003 Nov. 28; 55(12):1547-67].

Thus, according to one embodiment, the procollagen or telocollagen is expressed in a subcellular compartment of a plant that is devoid of endogenous P4H activity so as to avoid incorrect hydroxylation thereof. As is used herein, the phrase “subcellular compartment devoid of endogenous P4H activity” refers to any compartmentalized region of the cell which does not include plant P4H or an enzyme having plant-like P4H activity. According to one embodiment, the subcellular compartment is a vacuole.

Accumulation of the expressed procollagen in a subcellular compartment devoid of endogenous P4H activity can be effected via any one of several approaches.

For example, the expressed procollagen/telocollagen can include a signal sequence for targeting the expressed protein to a subcellular compartment such as the apoplast or an organelle (e.g. chloroplast). Examples of suitable signal sequences include the chloroplast transit peptide (included in Swiss-Prot entry P07689, amino acids 1-57) and the Mitochondrion transit peptide (included in Swiss-Prot entry P46643, amino acids 1-28).

Alternatively, the sequence of the procollagen can be modified in a way which alters the cellular localization of the procollagen when expressed in plants.

The present invention therefore contemplates genetically modified cells co-expressing both human procollagen and a P4H, capable of correctly hydroxylating the procollagen alpha chain(s) [i.e. hydroxylating only the proline (Y) position of the Gly-X-Y triplets]. P4H is an enzyme composed of two subunits, alpha and beta as set forth in Genbank Nos. P07237 and P13674. Both subunits are necessary to form an active enzyme, while the beta subunit also possesses a chaperon function.

The P4H expressed by the genetically modified cells of the present invention is preferably a human P4H which is encoded by, for example, SEQ ID Nos: 11 and 12. In addition, P4H mutants which exhibit enhanced substrate specificity, or P4H homologues can also be used. A suitable P4H homologue is exemplified by an Arabidopsis oxidoreductase identified by NCBI accession no: NP_{—}179363.

Since it is essential that P4H co-accumulates with the expressed procollagen chain, the coding sequence thereof is preferably modified accordingly (e.g. by addition or deletion of signal sequences).

In mammalian cells, collagen is also modified by Lysyl hydroxylase, galactosyltransferase and glucosyltransferase. These enzymes sequentially modify lysyl residues in specific positions to hydroxylysyl, galactosylhydroxylysyl and glucosylgalactosyl hydroxylysyl residues at specific positions. A single human enzyme, Lysyl hydroxylase 3 (LH3), as set forth in Genbank No. 060568, can catalyze all three consecutive modifying steps as seen in hydroxylysine-linked carbohydrate formation.

Thus, the genetically modified cells of the present invention may also express mammalian LH3. An LH3 encoding sequence such as that set forth by SEQ ID No: 13, can be used for such purposes.

The procollagen(s) and modifying enzymes described above can be expressed from a stably integrated or a transiently expressed nucleic acid construct which includes polynucleotide sequences encoding the procollagen alpha chains and/or modifying enzymes (e.g. P4H and LH3) positioned under the transcriptional control of functional promoters. Such a nucleic acid construct (which is also termed herein as an expression construct) can be configured for expression throughout the whole organism (e.g. plant, defined tissues or defined cells), and/or at defined developmental stages of the organism. Such a construct may also include selection markers (e.g. antibiotic resistance), enhancer elements and an origin of replication for bacterial replication.

There are various methods for introducing nucleic acid constructs into both monocotyledonous and dicotyledenous plants (Potrykus, I., Annu. Rev. Plant. Physiol., Plant. Mol. Biol. (1991) 42:205-225; Shimamoto et al., Nature (1989) 338:274-276). Such methods rely on either stable integration of the nucleic acid construct or a portion thereof into the genome of the plant, or on transient expression of the nucleic acid construct, in which case these sequences are not inherited by the plant's progeny.

In addition, several methods exist in which a nucleic acid construct can be directly introduced into the DNA of a DNA-containing organelle such as a chloroplast.

There are two principle methods of effecting stable genomic integration of exogenous sequences, such as those included within the nucleic acid constructs of the present invention, into plant genomes:
(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S, and Arntzen, C. J., Butterworth Publishers, Boston, Mass. (1989) p. 93-112.
(ii) Direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

There are various methods of direct DNA transfer into plant cells. In electroporation, protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals, tungsten particles or gold particles, and the microprojectiles are physically accelerated into cells or plant tissues.

Regardless of the transformation technique employed, once procollagen-expressing progeny are identified, such plants are further cultivated under conditions which maximize expression thereof. Progeny resulting from transformed plants can be selected, by verifying presence of exogenous mRNA and/or polypeptides by using nucleic acid or protein probes (e.g. antibodies). The latter approach enables localization of the expressed polypeptide components (by for example, probing fractionated plants extracts) and thus also verifies the plant's potential for correct processing and assembly of the foreign protein.

Following cultivation of such plants, the telopeptide-comprising collagen is typically harvested. Plant tissues/cells are preferably harvested at maturity, and the procollagen molecules are isolated using extraction approaches. Preferably, the harvesting is effected such that the procollagen remains in a state that it can be cleaved by protease enzymes. According to one embodiment, a crude extract is generated from the transgenic plants of the present invention and subsequently contacted with the protease enzymes.

As mentioned, the propeptide or telopeptide-comprising collagen may be incubated with a protease to generate atelocollagen or collagen prior to preparation of mesophase solutions. It will be appreciated that the propeptide or telopeptide-comprising collagen may be purified from the genetically engineered cells prior to incubation with protease, or alternatively may be purified following incubation with the protease. Still alternatively, the propeptide or telopeptide-comprising collagen may be partially purified prior to protease treatment and then fully purified following protease treatment. Yet alternatively, the propeptide or telopeptide-comprising collagen may be treated with protease concomitant with other extraction/purification procedures.

Exemplary methods of purifying or semi-purifying the telopeptide-comprising collagen of the present invention include, but are not limited to salting out with ammonium sulfate or the like and/or removal of small molecules by ultrafiltration.

According to one embodiment, the protease used for cleaving the recombinant propeptide or telopeptide comprising collagen is not derived from an animal. Exemplary proteases include, but are not limited to certain plant derived proteases e.g. ficin (EC 3.4.22.3) and certain bacterial derived proteases e.g. subtilisin (EC 3.4.21.62), neutrase. The present inventors also contemplate the use of recombinant enzymes such as rhTrypsin and rhPepsin. Several such enzymes are commercially available e.g. Ficin from Fig tree latex (Sigma, catalog #F4125 and Europe Biochem), Subtilisin from Bacillus licheniformis (Sigma, catalog #P5459) Neutrase from bacterium Bacillus amyloliquefaciens (Novozymes, catalog #PW201041) and TrypZean™, a recombinant human trypsin expressed in corn (Sigma catalog #T3449).

As used herein, the phrase “collagen fiber” refers to a non-soluble self-aggregate of collagen comprising a fibrous structure in which collagen molecules are packed in series and also in parallel. It will be appreciated that the collagen molecules may be in their monomeric form or their polymeric form. The collagen fibers generated according to the method of the present invention typically have a cross sectional diameter in the range of about 2 microns to 70 microns and more preferably between 5 microns and 30 microns.

As mentioned, the starting material for generating the fibers of the present invention is collagen (or procollagen) in a liquid crystal form.

Liquid crystal is a state of matter that is intermediate between the crystalline solid and the amorphous liquid. There are three basic phases of liquid crystals, known as smectic phase, nematic phase, and cholesteric phase and the present invention envisages the use of any of the above. In the smectic phase a one-dimensional translational order, as well as orientational order exists. In the nematic phase, only a long-range orientational order of the molecular axes exists. Cholesteric phase is also a nematic liquid type with molecular aggregates lie parallel to one another in each plane, but each plane is rotated by a constant angle from the next plane.

According to one embodiment, the liquid collagen solution is an acidic solution of collagen monomers (e.g. human or bovine collagen type I). Exemplary acids for solubilizing monomeric collagen include, but are not limited to hydrochloric acid (HCl) and acetic acid.

As used herein, the phrase “collagen monomers” refers to monomeric collagen that has not undergone the process of polymerization.

According to one embodiment a concentration of about 1 mM-100 mM HCl is used to solubilize the collagen monomers. An exemplary concentration of HCl which may be used to solubilize collagen monomers is about 10 mM HCl.

According to one embodiment a concentration of about 0.05 mM-50 mM acetic acid is used to solubilize the collagen monomers. An exemplary concentration of acetic acid which may be used to solubilize collagen monomers is about 0.5 M acetic acid.

The present invention contemplates addition of a crosslinker to the acidic solution of collagen monomers. The acidity of the solution prevents premature crosslinking. Following extrusion into a neutral coagulating solution, the crosslinker becomes activated and crosslinks the collagen fibrils. Examples of crosslinkers are further described herein below.

It will be appreciated that once the collagen is solubilized in the acid, the pH of the solution may be increased. The pH is selected such that the collagen therein still displays liquid crystal properties. Raising of the pH may be effected by dialyzing the acidic collagen against a higher pH buffer (e.g. pH 4/4.5 acetate buffer).

The present inventors have shown that when such a solution is extruded into a low phosphate buffer, this dope did not dissolve, and coagulated into a white, opaque fiber. The fiber maintained its shape and swelled substantially less then acidic dope fibers.

Generating solutions of liquid crystalline collagen monomers may be effected by concentrating a liquid collagen solution. The liquid collagen solution may be concentrated using any means known in the art, including but not limited to filtration, rotary evaporation and dialysis membrane.

Dialysis may be effected against a hygroscopic polymer, for example, PEG, a polyethylene oxide, amylose or sericin. Preferably, the PEG is of a molecular weight of 10,000-30,000 g/mol and has a concentration of 25-50%. According to a particular embodiment, a slide-a-lyzer dialysis cassette (Pierce, MW CO 3500) is used. Typically, the dialysis is effected in the cold (e.g. at about 4° C.). The dialysis is effected for a time period sufficient to result in a final concentration of aqueous collagen solution of about 10 mg/ml or more. According to one embodiment, the solution of monomeric collagen is at a concentration of about 100-200 mg/ml or between 0.7-0.3 mM.

In most cases dialysis for 2-16 hours is sufficient, depending on volume and concentration.

According to another embodiment, the solution of liquid crystalline collagen comprises high concentrations (5-30 mg/ml, depending on the collagen type) of procollagen molecules in physiological buffer. It has been shown that such solutions develop long range nematic and precholesteric liquid crystal ordering extending over 100 μm² domains, while remaining in solution (R. Martin et al., J. Mol. Biol. 301: 11-17 (2000)). Procollagen concentrations in vivo are estimated at several tens of milligrams per milliliter in the secretory vesicles and the molecules are often observed to be aligned in a nematic-like ordering.

In another embodiment, the starting collagen material may be prepared by ultrasonic treatment. Brown E. M. et al. Journal of American Leather Chemists Association, 101:274-283 (2006), herein incorporated by reference by its entirety.

The solutions of liquid crystalline collagen may comprise additives such as ATP to decrease the threshold of the required concentration to develop the liquid crystal state. Without being bound by any particular theory, generally, highly negative charged molecules (more that −3) can be used as additives to the collagen solution to promote the orientation or adhesion of the collagen, so that the collagen can form liquid crystals at relatively lower concentration. Suitable additives include, but are not limited to ATP, vanadate, insulin, phosphate and VGF.

Other additives that may be added to the starting material of the present invention include antimicrobials such as silver nitrate, iodized radicals (e.g., Triosyn®; Hydro Biotech), benzylalkonium chloride, alkylpyridinium bromide (cetrimide), and alkyltrimethylammonium bromide. Viscosity enhancers may be added to improve the rheological properties of the starting material. Examples include, but are not limited to polyacrylates, alginate, cellulosics, guar, starches and derivatives of these polymers, including hydrophobically modified derivatives.

The present invention further contemplates addition of hyaluronic acid (HA) to the solution of liquid crystalline collagen to generate a highly extensible and spinable dope.

As mentioned, the collagen fibers of the present invention are generated by extruding the solution of liquid crystalline collagen into a coagulating solution.

As used herein, the term “extruding” as used herein refers to the forcing of a flowable material out through a relatively narrow aperture (i.e. a nozzle in the widest sense), e.g. through a needle.

According to one embodiment the aperture has an inner diameter of about 10-100 gauge (ga) (e.g. about 30 ga).

According to another embodiment, the extruding is effected using a spinneret. The spinneret can have a single orifice or multiple orifices, depending on, for example, the volume of collagen solution to be spun, and the number of collagen fibers to be produced. Spinnerets may be composed of various materials, including metals and alloys, such as stainless steel or tantalum, polymeric materials, such as PEEK tubing, ceramics or carbon-composite materials. Spinnerets with a single orifice may be made of metal, preferably stainless steel. Spinnerets with multiple orifices are preferably made of polymeric tubing, most preferably PEEK tubing. Spinnerets may also be treated with substances, such as TEFLON™ or spray silicon, in such a manner as to prevent adherence of the dope to the spinneret needle.

The coagulating solution serves to stabilize or preserve the molecular orientation of the extruded collagen molecules. Typically, the stabilizing agent in the coagulating solution is at a high enough osmolarity such that is can extract water from the collagen mesophase and dry it.

According to one embodiment of this aspect of the present invention, the coagulating solution comprises an organic solvent. The present invention contemplates coagulating solutions wherein at least 50% thereof comprises the organic solvent. The present invention further contemplates coagulating solutions wherein at least 70% thereof comprises the organic solvent. The present invention further contemplates coagulating solutions wherein at least 90% thereof comprises the organic solvent.

The collagen typically remains in the coagulating solution for at least 15 minutes.

Exemplary organic solvents that may be used according to this aspect of the present invention include, but are not limited to acetone, methanol, isopropanol, methylated spirit and ethanol.

Alternatively, the coagulating solution may be a concentrated aqueous salt solution having a high ionic strength. The high osmotic pressure of a concentrated salt solution draws the water away from the collagen protein, thereby facilitating fiber coagulation. Preferred coagulating solutions include aqueous solutions containing a high concentration of aluminum sulfate, ammonium sulfate, sodium sulfate, or magnesium sulfate. Additives, particularly acids, such as acetic acid, sulfuric acid, or phosphoric acid, or also sodium hydroxide may be added to the salt-based coagulation bath.

Contemplated salt coagulating solutions may comprise one or more salts of high solubility such as, for example, salts containing one or more of the following anions: nitrates, acetates, chlorates, halides (fluoride, chloride, bromide, iodide), sulfates, sulfides, sulfites, carbonates, phosphates, hydroxides, thiocyanates, bicarbonates, formates, propionates, and citrates; and one or more of the following cations: ammonium, aluminum, calcium, cesium, potassium, lithium, magnesium, manganese, sodium, nickel, rubidium, antimony, and zinc. The solution may also contain an acid of the same anion as the salt, e.g., nitric, acetic, hydrochloric, sulfuric, carbonic, phosphoric, formic, propionic, citric, or lactic acid, or another acid which also forms highly soluble salts with the cation(s) present. Preferably, the salts used in the coagulating solution of the present invention are multivalent anions and/or cations, resulting in a greater number of ions, and proportionally higher ionic strength, on dissociation. Typically, concentrated salt coagulating solutions comprise about 30%-70% (w/v) of salt; preferably about 40-65%.

According to another embodiment the coagulation solution is a solution that allows polymerization (i.e. fibrilogenesis) of collagen monomers. Such a solution typically is at a neutral or high pH (e.g. pH 7.4 or more) to allow for polymerization. An exemplary fibrilogenesis buffer comprises between about 5 mM sodium phosphate to about 50 mM sodium phosphate.

Useful additives may be included in the coagulating medium include, but are not limited to surfactants, osmoprotective agents, stabilizing agents, UV inhibitors, and antimicrobial agents. Stabilizers that protect against UV radiation, radical formation, and biodegradation include, for example, 2-hydroxybenzophenones, 2-hydroxyphenyl-2-(2H)-benzotriazoles, cifmamates, and mixtures thereof. These chemicals are capable of absorbing and dissipating UV energy, thereby inhibiting UV degradation. Free radicals are neutralized by hindered amine light stabilizers (HALS), butylated hydroxyanisole (BHA), and butylated hydroxytoluene (BHT).

The growing fiber can be extruded through an air gap before entering the coagulation medium, or the fiber can be extruded directly into the coagulation medium. Additionally, the fiber may be processed through one or more (e.g., two, three, four or five) coagulation baths, preferably of the same composition, to extend the residence time in the bath, or, in certain embodiments, of sequentially lesser coagulant concentrations, optionally followed by one or more rinse/wash baths.

Following extrusion into the coagulating solution of the present invention, the fibers are pulled out of the solution (i.e. isolated). According to one embodiment the fibers are drawn from the solution. Such a process typically improves the axial orientation and toughness of the collagen fiber. The drawing process can develop end-use properties such as modulus and tenacity. The fibers are typically stretched or drawn under conditions wherein significant molecular orientation is imparted. The variables include but are not limited to draw ratio, temperature and strain rate.

Drawing may be effected using a set of godets, with the filament wrapped several times (e.g. 3-8 times) around the chromium roller of each godet.

The fibers may be optionally washed in one or more wash baths following the drawing stage. In addition, following the drawing stage, the fibers may be dried or dehydrated to evaporate the coagulating solution. Alternatively, the fibers may be washed in baths of successively lower concentration of the coagulant used, e.g., successively lower organic solvent concentrations subsequent to an organic solvent-based coagulant bath, until an ultimate water bath.

It will be appreciated that if the collagen fibers have not been extruded directly into a fibrillogenesis buffer, the fibers may be incubated in this buffer following drawing. This would ensure an even higher degree of stability and structure.

Alternatively, or additionally, the extruded collagen may be crosslinked using any one of the below methods: 1. by glutaraldehyde and other chemical crosslinking agents; 2. by glycation using different sugars; 3. by Fenton reaction using metal ions such as copper; 4. by lysine oxidase; or 5. by UV radiation.

Following generation and optional crosslinking/polymerization, the physical properties of the collagen fibers may be tested.

To measure such physical properties, any suitable apparatus having (1) two clamps for attaching to the fiber(s), (2) a force transducer attached to one of the clamps for measuring the force applied to the fiber, (3) a means for applying the force, and (4) a means for measuring the distance between the clamps, is suitable. For example, tensiometers can be purchased from manufacturers MTS, Instron, and Cole Parmer. To calculate the tensile strength, the force at failure is divided by the cross-sectional area of the fiber through which the force is applied, resulting in a value that can be expressed in force (e.g., Newtons) per area. The stiffness is the slope of the linear portion of the stress/strain curve. Strain is the real-time change in length during the test divided by the initial length of the specimen before the test begins. The strain at failure is the final length of the specimen when it fails minus the initial specimen length, divided by the initial length.

An additional physical property that is associated with the extent of cross-linking in a composition is the shrinkage temperature. In general, the higher the temperature at which a collagenous composition begins to shrink, the higher the level of cross-linking. The shrinkage temperature of a fiber can be determined by immersing the fiber in a water or buffer bath, raising the temperature of the water or buffer bath, and observing the temperature of the water or buffer bath at which the fiber shrinks. In order to observe shrinkage, a tension may be applied on the fiber.

According to one embodiment, the fibers generated according to the method of the present invention comprise a stiffness between about 0.3 Gpa and 5 Gpa.

According to one embodiment, the fibers generated according to the method of the present invention comprise a tensile strength between about 100-500 MPa.

Techniques for directing or casting the collagen fibers generated according to the methods of the present invention for manufacturing of aligned collagen matrices into 2D or 3D structures are widely known and include for example alignment by surface templating [David A. Cisneros, Jens Friedrichs, Anna Taubenberger, Clemens M. Franz, and Daniel J. Muller. Creating Ultrathin Nanoscopic Collagen Matrices For Biological And Biotechnological Applications small 2007, 3, No. 6, 956-963]; by chemical patterning [Frederic A. Denis, Antoine Pallandre, Bernard Nysten, Alain M. Jonas, and Christine C. Dupont-Gillain. Alignment and Assembly of Adsorbed Collagen Molecules Induced by Anisotropic Chemical Nanopatterns. small 2005, 1, No. 10, 984-991]; nanolithography [Donna L. Wilson, Raquel Martin, Seunghun Hong, Mark Cronin-Golomb, Chad A. Mirkin, and David L. Kaplan. Surface organization and nanopatterning of collagen by dip-pen nanolithography. Proc Natl Acad Sci USA. 2001 Nov. 20; 98(24):13660-4]; electrochemical fabrication [Xingguo Cheng, Umut A. Gurkan, Christopher J. Dehen, Michael P. Tate, Hugh W. Hillhouse, Garth J. Simpson, Ozan Akkus. An electrochemical fabrication process for the assembly of anisotropically oriented collagen bundles. Biomaterials 29 (2008) 3278-3288]; magnetic field [Jim Torbet, Marilyne Malbouyres, Nicolas Builles, Virginie Justin, Muriel Roulet, Odile Damour, Ake Oldberg, Florence Ruggiero, David J. S. Hulmes. Orthogonal scaffold of magnetically aligned collagen lamellae for corneal stroma reconstruction. Biomaterials 28 (2007) 4268-4276]; and by shear flow [Babette Lanfer, Uwe Freudenberg, Ralf Zimmermann, Dimitar Stamov, Vincent Ko\ddot{}rber, Carsten Werner. Aligned fibrillar collagen matrices obtained by shear flow deposition. Biomaterials 29 (2008) 3888-3895].

The collagen fibers generated according to the method of the present invention may be used per se, or as part of a composite material. The components of the composites of the present invention may be attached to, coated on, embedded or impregnated into the collagen of the present invention. In such composites, the collagen may be uncrosslinked, partially crosslinked or fully crosslinked. Exemplary components of the composite material include, but are not limited to minerals, pharmaceutical agents (i.e. drugs) polysaccharides and polypeptides.

Exemplary polysaccharides that may be used in composite materials of the present invention include, but are not limited to glycosaminoglycans such as chondroitin sulfate of type A, C, D, or E, dermatan sulfate, keratan sulfate, heparan sulfate, heparin, hyaluronic acid and their derivatives, individually or mixed.

Exemplary polypeptides that may be used in composite materials of the present invention include, but are not limited to resilin, silk, elastin and fibronectin.

Exemplary minerals that may be used in composite materials of the present invention include, but are not limited to calcium, magnesium, boron, zinc, copper, manganese, iron, silicon, selenium, phosphorus and sulfur. Methods for preparing collagen mineral composites are well known in the art, see for example WO/2006/118803.

The collagen fibers generated according to the method of the present invention showed very strong extinction patterns. This attributes to the high degree of intrafibrillar order in the fiber. It is therefore anticipated that the collagen fibers show superior mechanical properties compared to those that have not been generated by direct extrusion into organic solvents from a mesophase starting material.

Since the collagen of the present invention has been shown to be highly structured and comprise high strength, the collagen may be particularly suitable for bioprostheses suitable for tendon and/or ligament repair, augmentation, and/or replacement. A biomaterial with increased strength over that of natural tissue (muscle and the like) can allow for a bioprosthesis that has a smaller cross-sectional area than that of the natural tissue being replaced or repaired. The smaller area can improve the function of the bioprosthesis as a scaffold for neo-tendon or ligament in-growth, which may augment strength and/or long term survival rate of the repair. The use of high-strength fibers on medical devices and constructs may also offset or reduce the effects of stress concentration factors that reside at regions of integration in adjacent tissue such as bone.

The collagen generated according to the method of the present invention or composites thereof may therefore also be used as part of a scaffold.

As used herein, the term “scaffold” refers to a 3D matrix upon which cells may be cultured (i.e., survive and preferably proliferate for a predetermined time period).

The scaffold may be fully comprised of the collagen of the present invention or composites thereof, or may comprise a solid support on which is layered the collagen of the present invention.

A “solid support,” as used refers to a three-dimensional matrix or a planar surface (e.g. a cell culture plate) on which cells may be cultured. The solid support can be derived from naturally occurring substances (i.e., protein based) or synthetic substances. Suitable synthetic matrices are described in, e.g., U.S. Pat. Nos. 5,041,138, 5,512,474, and 6,425,222. For example, biodegradable artificial polymers, such as polyglycolic acid, polyorthoester, or polyanhydride can be used for the solid support. Calcium carbonate, aragonite, and porous ceramics (e.g., dense hydroxyapatite ceramic) are also suitable for use in the solid support. Polymers such as polypropylene, polyethylene glycol, and polystyrene can also be used in the solid support.

Therapeutic compounds or agents that modify cellular activity can also be incorporated (e.g. attached to, coated on, embedded or impregnated) into the scaffold material or a portion thereof. In addition, agents that act to increase cell attachment, cell spreading, cell proliferation, cell differentiation and/or cell migration in the scaffold may also be incorporated into the scaffold. Such agents can be biological agents such as an amino acid, peptides, polypeptides, proteins, DNA, RNA, lipids and/or proteoglycans.

Suitable proteins which can be used along with the present invention include, but are not limited to, extracellular matrix proteins [e.g., fibrinogen, collagen, fibronectin, vimentin, microtubule-associated protein 1D, Neurite outgrowth factor (NOF), bacterial cellulose (BC), laminin and gelatin], cell adhesion proteins [e.g., integrin, proteoglycan, glycosaminoglycan, laminin, intercellular adhesion molecule (ICAM) 1, N-CAM, cadherin, tenascin, gicerin, RGD peptide and nerve injury induced protein 2 (ninjurin2)], growth factors [epidermal growth factor, transforming growth to factor-α, fibroblast growth factor-acidic, bone morphogenic protein, fibroblast growth factor-basic, erythropoietin, thrombopoietin, hepatocyte growth factor, insulin-like growth factor-I, insulin-like growth factor-II, Interferon-β, platelet-derived growth factor, Vascular Endothelial Growth Factor and angiopeptin], cytokines [e.g., M-CSF, IL-1beta, IL-8, beta-thromboglobulin, EMAP-II, G-CSF and IL-10], proteases [pepsin, low specificity chymotrypsin, high specificity chymotrypsin, trypsin, carboxypeptidases, aminopeptidases, proline-endopeptidase, Staphylococcus aureus V8 protease, Proteinase K (PK), aspartic protease, serine proteases, metalloproteases, ADAMTS17, tryptase-gamma, and matriptase-2] and protease substrates.

Additionally and/or alternatively, the scaffolds of the present invention may comprise an antiproliferative agent (e.g., rapamycin, paclitaxel, tranilast, Atorvastatin and trapidil), an immunosuppressant drug (e.g., sirolimus, tacrolimus and Cyclosporine) and/or a non-thrombogenic or anti-adhesive substance (e.g., tissue plasminogen activator, reteplase, TNK-tPA, glycoprotein IIb/IIIa inhibitors, clopidogrel, aspirin, heparin and low molecular weight heparins such as enoxiparin and dalteparin).

Cells which may be seeded on the collagen of the present invention may comprise a heterogeneous population of cells or alternatively the cells may comprise a homogeneous population of cells. Such cells can be for example, stem cells (such as embryonic stem cells, bone marrow stem cells, cord blood cells, mesenchymal stem cells, adult tissue stem cells), progenitor cells, or differentiated cells such as chondrocytes, osteoblasts, connective tissue cells (e.g., fibrocytes, fibroblasts and adipose cells), endothelial and epithelial cells. The cells may be naïve or genetically modified.

According to one embodiment of this aspect of the present invention, the cells are mammalian in origin.

Furthermore, the cells may be of autologous origin or non-autologous origin, such as postpartum-derived cells (as described in U.S. application Ser. Nos. 10/887,012 and 10/887,446). Typically the cells are selected according to the tissue being generated.

Techniques for seeding cells onto or into a scaffold are well known in the art, and include, without being limited to, static seeding, filtration seeding and centrifugation seeding.

It will be appreciated that to support cell growth, the cells are seeded on the collagen of the present invention in the presence of a culture medium.

The culture medium used by the present invention can be any liquid medium which allows at least cell survival. Such a culture medium can include, for example, salts, sugars, amino acids and minerals in the appropriate concentrations and with various additives and those of skills in the art are capable of determining a suitable culture medium to specific cell types. Non-limiting examples of such culture medium include, phosphate buffered saline, DMEM, MEM, RPMI 1640, McCoy's 5A medium, medium 199 and IMDM (available e.g., from Biological Industries, Beth Ha'emek, Israel; Gibco-Invitrogen Corporation products, Grand Island, N.Y., USA).

The culture medium may be supplemented with various antibiotics (e.g., Penicillin and Streptomycin), growth factors or hormones, specific amino acids (e.g., L-glutamin) cytokines and the like.

The scaffolds of the present invention may be administered to subjects in need thereof for the regeneration of tissue such as connective tissue, muscle tissue such as cardiac tissue and pancreatic tissue. Examples of connective tissues include, but are not limited to, cartilage (including, elastic, hyaline, and fibrocartilage), collagen, adipose tissue, reticular connective tissue, embryonic connective tissues (including mesenchymal connective tissue and mucous connective tissue), tendons, ligaments, and bone.

The collagen fibers generated according to the method of the present invention may be used to prepare films and matrices. According to one embodiment, biomedical devices may be formed from such films and matrices.—e.g. collagen membranes for hemodialysis.

According to one embodiment, the collagen films and membranes are kept cold, in the dark in the dark or hydrated in order to prevent the collapsing and condensation of the structure.

According to another embodiment, the collagen generated according to the method of the present invention (or films derived therefrom) is used in cell cultures. Collagen as a film or as a coating on other materials has also been used in tissue culture for the growth of fastidious cells. The protein surface and the orientation of the fibers appear to promote cell growth in vitro and probably in vivo as well.

The phrase “cell culture” or “culture” as used herein refers to the maintenance of to cells in an artificial, e.g., an in vitro environment. It is to be understood, however, that the term “cell culture” is a generic term and may be used to encompass the cultivation not only of individual prokaryotic (e.g., bacterial) or eukaryotic (e.g., animal, plant and fungal) cells, but also of tissues, organs, organ systems or whole organisms.

Generally, cell culture is carried out by growing cells in a culture vessel in the presence of cell culture medium. By “culture vessel” herein is meant a glass, plastic, or metal container and the like that can provide an aseptic environment for culturing cells. Culture vessels include but are not limited to petri dishes and 96-well plates.

In some embodiments, the collagen generated according to the method of the present invention is used to coat the surface of a cell culture vessel.

In some embodiments, the collagen generated according to the method of the present invention is used in a wound healing process. During the wound healing process, oriented collagen acts to modulate cell proliferation and migration and is important in the wound contraction process. Cuttle L., et al., Wound Repair and Regeneration, 13:198-204 (2005).

In some embodiments, collagen films provided herein are used to prevent adhesions following tendon injuries, to lengthen levator palpebrae muscles ophthalmic surgery, and to repair transected nerves. Collagen films provided herein may further be used for burn dressings and wound healing. In some embodiments, the collagen is preferably not heavily cross-linked. If the films are heavily cross-linked, they do not become incorporated into the tissue, but rather, granulation, and re-epithelialization take place beneath the films. Here the film acts as an inert dressing. Collagen felt or sponge, on the other hand, may function as a true artificial skin. Healing of bone defects and wounds also appears enhanced by collagen.

It will be appreciated that the collagen of the present invention comprises a myriad of uses other than for tissue regeneration including, but not limited to treatment of diseases such as interstitial cystitis, scleroderma, and rheumatoid arthritis cosmetic surgery, as a healing aid for burn patients, as a wound-healing agent, as a dermal filler, for spinal fusion procedures, for urethral bulking, in duraplasty procedures, for reconstruction of bone and a wide variety of dental, orthopedic and surgical purposes.

The collagen of the present invention may be formulated as pharmaceutical and/or cosmetic compositions.

The term “cosmetic composition” as used herein refers to a composition formulated for external application to human or animal skin, nails, or hair for the purpose of beautifying, coloring, conditioning, or protecting the body surface. The present cosmetic composition can be in any form including for example: a gel, cream, lotion, makeup, colored cosmetic formulations, shampoo, hair conditioner, cleanser, toner, aftershave, fragrance, nail enamel, and nail treatment product.

The phrase “colored cosmetic formulation” refers to cosmetics containing pigment including for example eye shadow, lipsticks and glosses, lip and eye pencils, mascara, and blush.

For example, the collagen fibers of the present invention may also be used as a cosmetic agent for treatment of skin and hair.

Thus, the present invention contemplates the collagen of the present invention as a substance which can be topically applied, optionally in combination with other active substance such as for example a vitamin (vitamin A, C, E or their mixtures) or other topically active substances including but not limited to avarol, avarone or plant extracts, such as Extr. Cepae or Extr. Echinaceae pallidae. The collagen of the present invention may be formulated as a topical agent in the form of creams, ointments, lotions or gels such as a hydrogels e.g. on the basis of polyacrylate or an oleogel e.g. made of water and Eucerin.

Oleogels comprising both an aqueous and a fatty phase are based particularly on Eucerinum anhydricum, a basis of wool wax alcohols and paraffin, wherein the percentage of water and the basis can vary. Furthermore additional lipophilic components for influencing the consistency can be added, e.g. glycerin, polyethylene glycols of different chain length, e.g. PEG400, plant oils such as almond oil, liquid paraffin, neutral oil and the like. The hydrogels of the present invention can be produced through the use of gel-forming agents and water, wherein the first are selected especially from natural products such as cellulose derivatives, such as cellulose ester and ether, e.g. hydroxyethyl-hydroxypropyl derivatives, e.g. tylose, or also from synthetic products such as polyacrylic acid derivatives, such as Carbopol or Carbomer, e.g. P934, P940, P941. They can be produced or polymerized based on known regulations, from alcoholic suspensions by adding bases for gel formation.

Exemplary amounts of collagen in the gel include 0.01-30 g per 100 g of gel, 0.01-10 g per 100 g of gel, 0.01-8 g per 100 g of gel, 0.1-5 g per 100 g of gel.

The cosmetic composition may comprise other agents capable of conditioning the body surface including, for example humectants; emollients; oils including for example mineral oil; and shine enhancers including for example dimethicone and cyclomethicone. The present conditioning agents may be included in any of the present pharmacological and/or cosmetic compositions.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the collagen accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated to sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection to suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (collagen) effective to prevent, alleviate or ameliorate symptoms of a disorder (e.g., skin disease).

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Generation of Fibers from Collagen Mesophase

Material and Methods

Generation of Fibers:

Acid soluble (10 mm HCl) bovine collagen at 3 mg/ml was purchased from Inamed (PURECELL™). The collagen was dialyzed in the cold against a 40% solution of 20,000 MW Polyethylene Glycol (PEG) using 3.5 kDa dialysis tubing in 10 mM HCl pH 2.

The collagen mesophase was extruded through a 30 ga needle into the coagulation medium. Organic solvents used as coagulation medium included acetone, ethanol and isopropanol.

Other coagulation mediums which were tested are listed below:

Buffer 1 (pH 8)—high osmolarity coagulation buffer

16 gr (113 mM) Sodium phosphate dibasic

6.9 gr (57 mM) Tris

7.9 gr (135 mM) Sodium Chloride

20% PEG

Buffer 1 and Subsequent Incubation in PBS at 34° C. for 48 Hours:

PBS (pH 7.4):

138 mM NaCl

10 mM Phosphate

2.7 mM KCl

Buffer 2 (pH 7.55)—high ionic strength buffer

20% (˜4M) Sodium Chloride

118 mM Sodium phosphate dibasic

pH 7.55

### Buffer 3 (pH 7.1)

75 mM Sodium Chloride

6 mM Sodium phosphate dibasic

For the morphological fiber studies, the mesophase collagen samples were compared with a non-mesophase collagen sample extruded into fibrillogenesis buffer.

For the mechanical studies, mesophase collagen samples (including mesophase collagen extruded into ethanol; mesophase collagen extruded into acetone; mesophase collagen extruded into isopropanol; and mesophase collagen extruded into fibrillogenesis buffer) were compared with dry rat tail tendon.

Extruded fibers were analyzed under a light microscope and an electron microscope.

Mechanical Testing:

Dry fibers were stretched until failure in an “Instron” apparatus.

Fibrillogenesis Buffer (FB):

135 mM NaCl 30 mM TrizmaBase (Tris), and 5 mM sodium phosphate dibasic, pH 7.4, as described in Pins et al., Biophysical Journal Volume 73 Oct. 1997 2164-2172 (in some cases 40% 20,000 MW Polyethylene Glycol (PEG) was added). Following coagulation in FB, the fibers were later dehydrated in 50% ethanol.

Results

Morphological Studies of Fibers:

Under polarized light microscopy, fibers exhibited birefringent bands (FIG. 1A) perpendicular to the fiber axis (the injection axis) that are reminiscent of bands displayed by rat tail tendon (RTT) and chick extensor tendon. The banding is attributed to the wave like structures (WLS) or crimp displayed by tendon fibrils in vivo.

Collagen mesophase (100 mg/ml) extruded into isopropanol (FIGS. 1A-B) shows a much stronger extinction pattern compared to 10 mg/ml collagen solution (not a mesophase) extruded into fibrillogenesis buffer and dried in isopropanol [FIG. 1C—Kato et al., J Bone Joint Surg Am. 1991; 73:561-574]. The stronger extinction pattern is attributed to the higher degree of intrafibrillar order present in the mesophase extruded fiber relative to the soluble collagen extruded fibers.

Under an electron microscope, it is evident that the extruded fibers are composed of longer and better aligned fibrils compared to dilute collagen extruded fibers, and that they show higher resemblance to natural tendon (FIGS. 2A-F).

Injection into different coagulation solutions and addition of an incubation stage generated collagen fibers of different morphologies—especially diameter of fibrils and their density of packing.

Acetone:

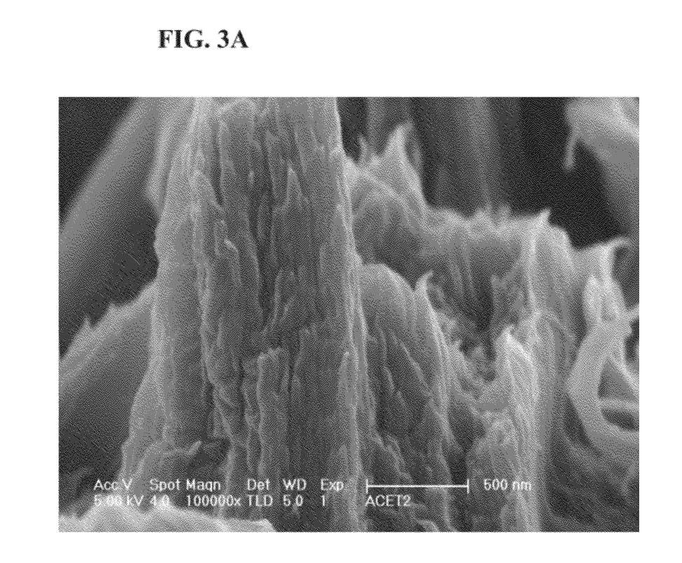

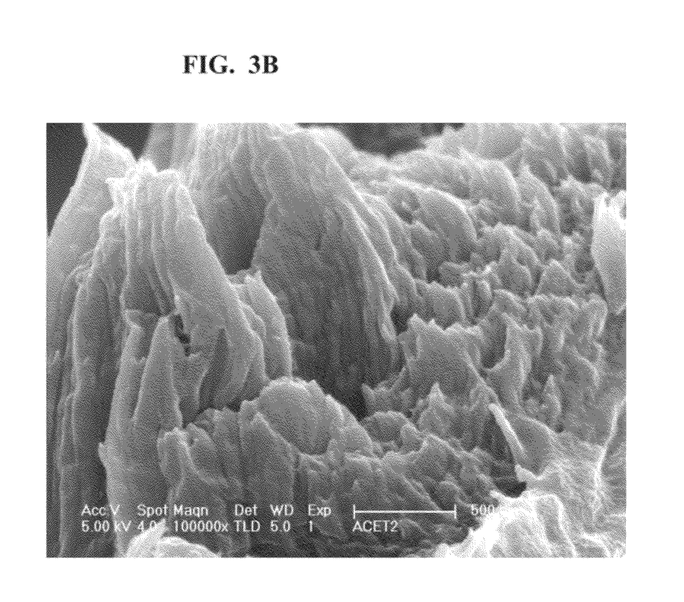

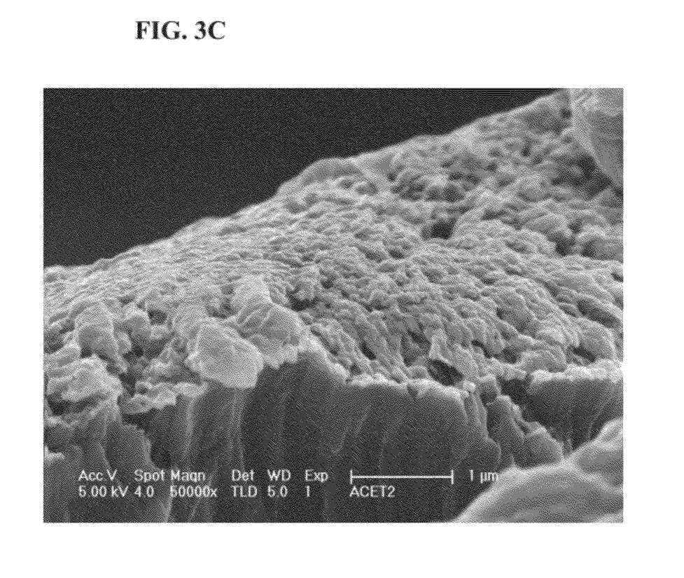

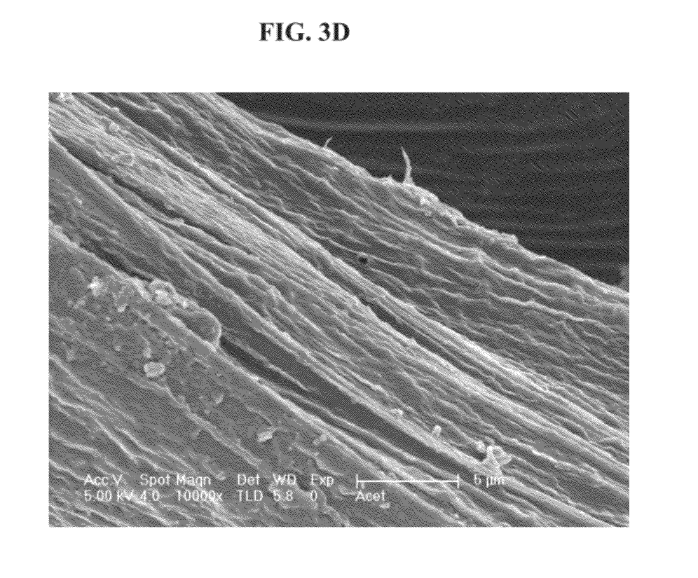

Injection of mesophase collagen into acetone dehydrated the dope the most quickly out of all the coagulation solutions tested. The fibers turned completely white in about 10 to 20 seconds, and their diameter was the smallest. The fibers were crisp, hard to touch and stiff—not elastic. When the fibers were brought out of the solvent they dried very fast. As illustrated in FIG. 3D, the outer layer of the fiber were tightly fused, sealed and shrunken. The fibrils were tightly packed and fused to together (FIGS. 3B-C), and no D banding was observed (FIG. 3A).

Isopropanol:

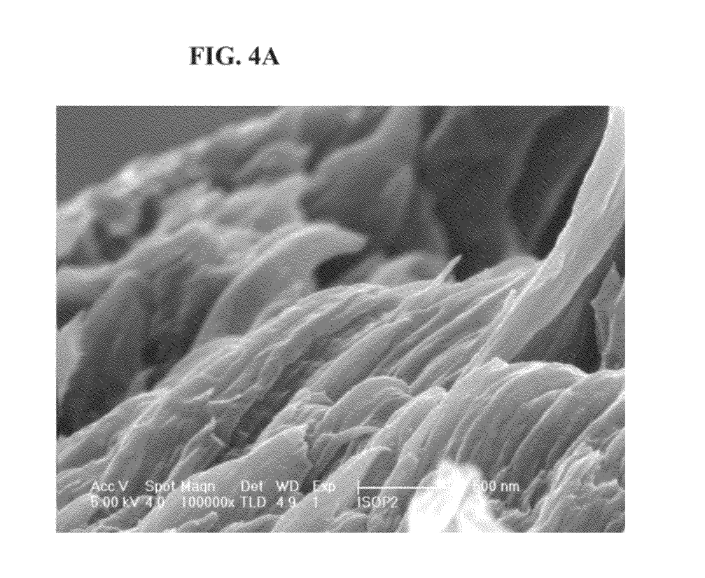

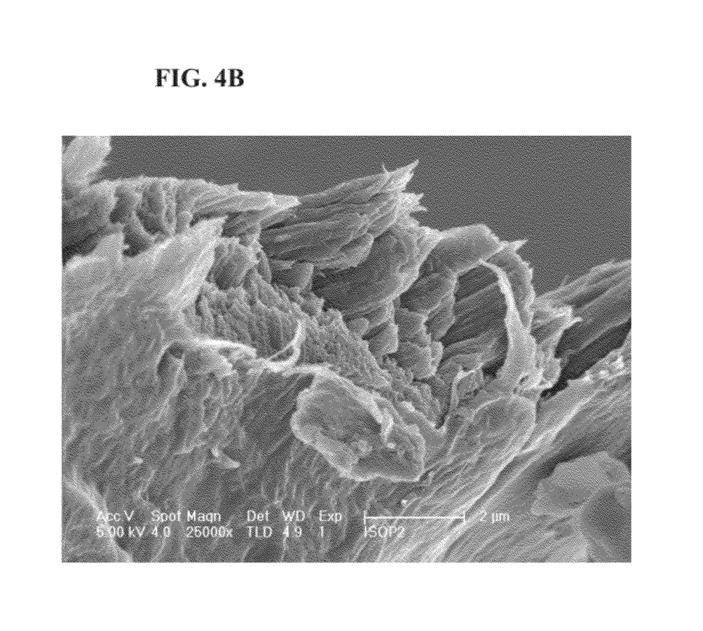

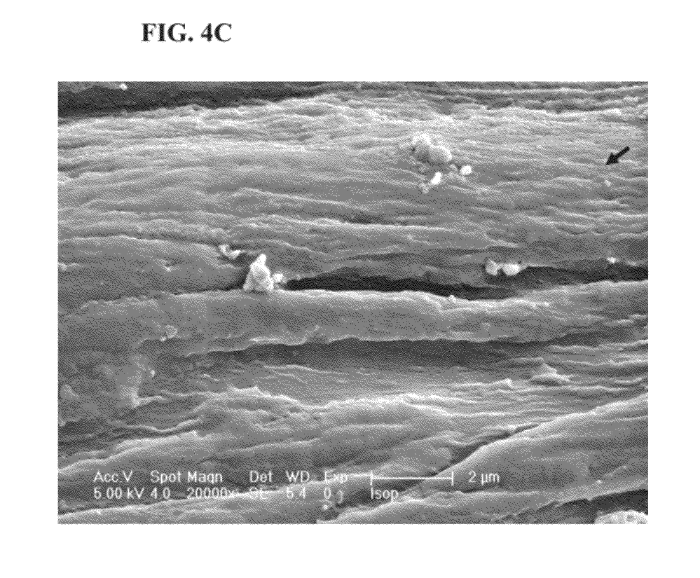

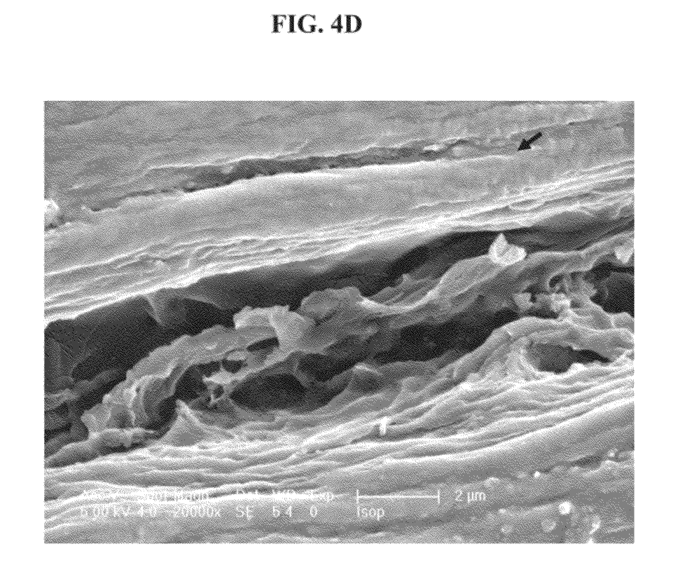

Injection of mesophase collagen into isopropanol generated fibers similar in appearance to acetone, although the dehydration time was slightly slower. The fibers turned white and stiff—not elastic. As illustrated in FIGS. 4C-D, the outer layer of the fiber appeared to be fused and sealed, but fibrilar forms could be identified (arrows). The fibrils were partially fused, but more clearly defined (FIGS. 4 B,C), no D banding is observed (FIG. 4A).

Ethanol:

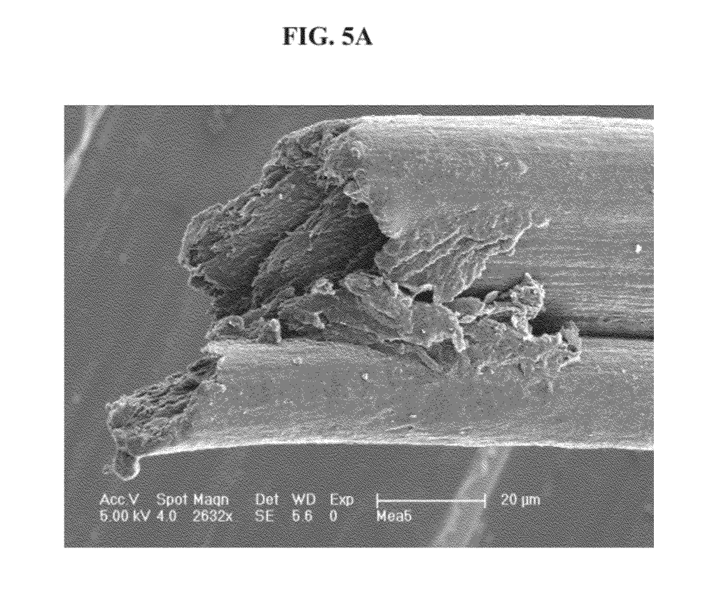

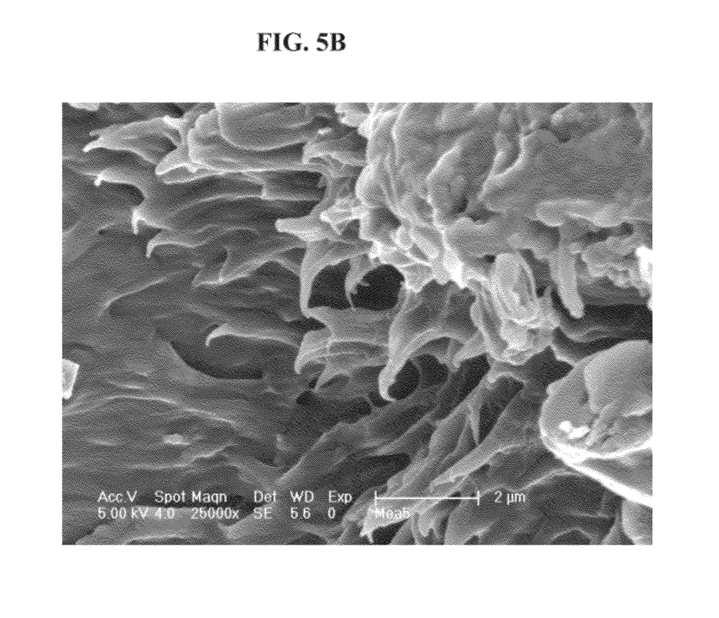

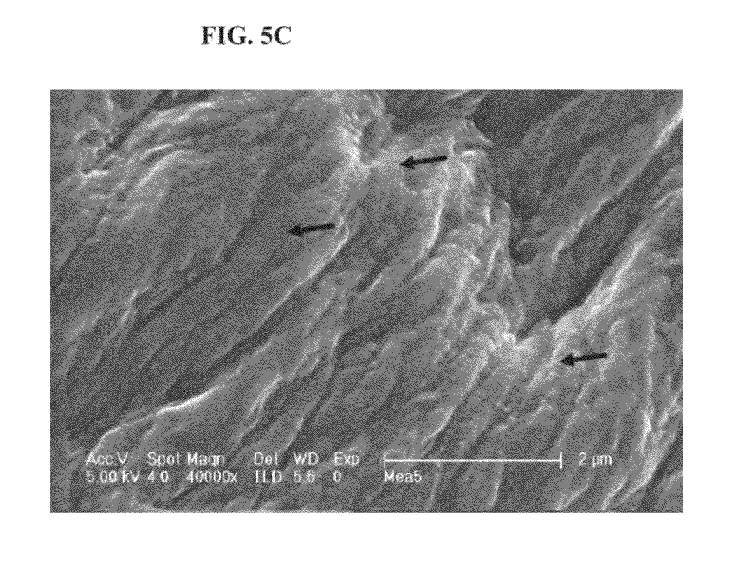

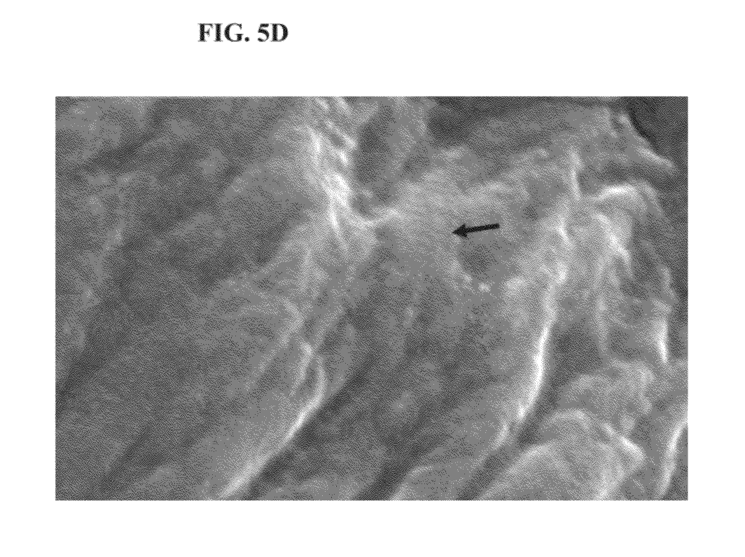

Injection of mesophase collagen into ethanol generated white/opaque fibers, which were partially transparent. Dehydration time was about five minutes. The fibers were not crisp, but softer and more elastic compared to isopropanol and acetone. As illustrated in FIGS. 5C-D, the outer layer of the fiber appeared formed from fused fibrils. Extensive D banding was observed (FIGS. 5C-D, arrows). The fibrils inside the fibers appeared to be more separate and defined (FIGS. 5A-B).

Buffer 1:

Injection of mesophase collagen into buffer 1 generated swollen and flexible fibers.

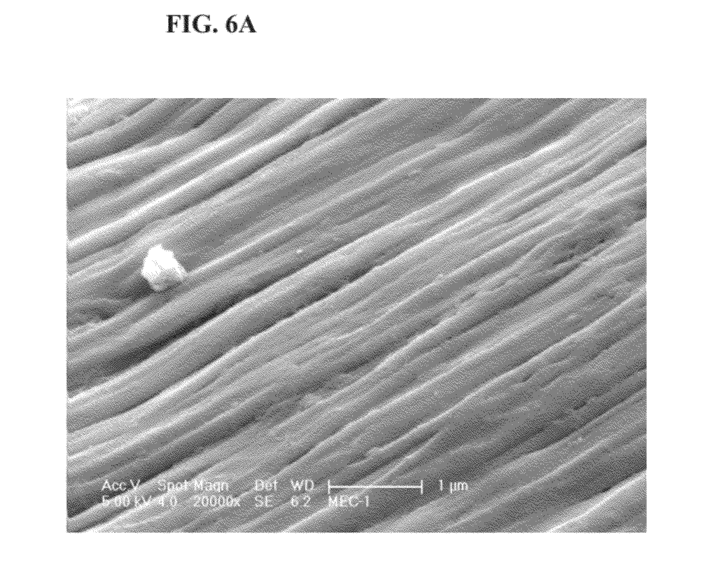

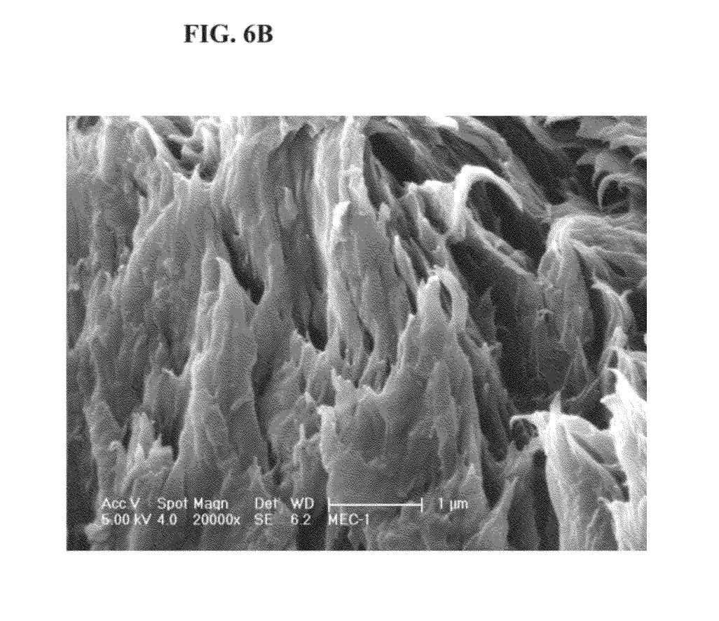

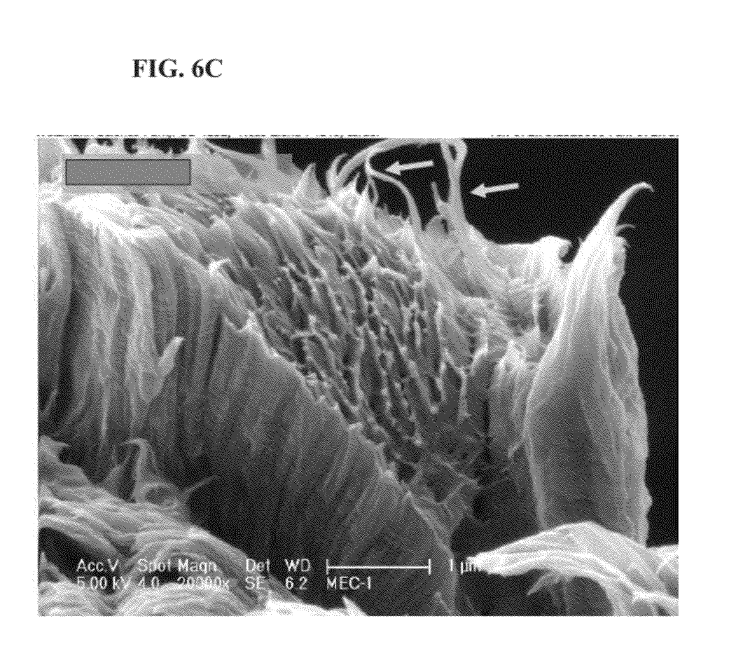

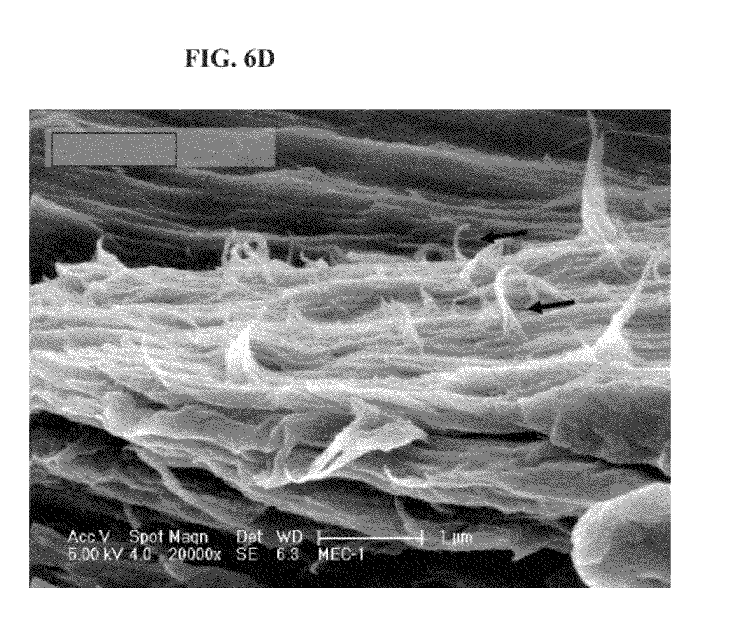

As illustrated in FIG. 6A, the fiber surface showed clear notable fibrilar forms (FIG. 6A). The fibrils inside the fiber were large in diameter (>100 [nm]) and fused together extensively, sometimes eliminating the fibrilar shape altogether (FIG. 6B). Only a few separate fibrils could be seen, and no D banding (arrows, FIGS. 6C-D).

Buffer 1 and subsequent incubation in PBS

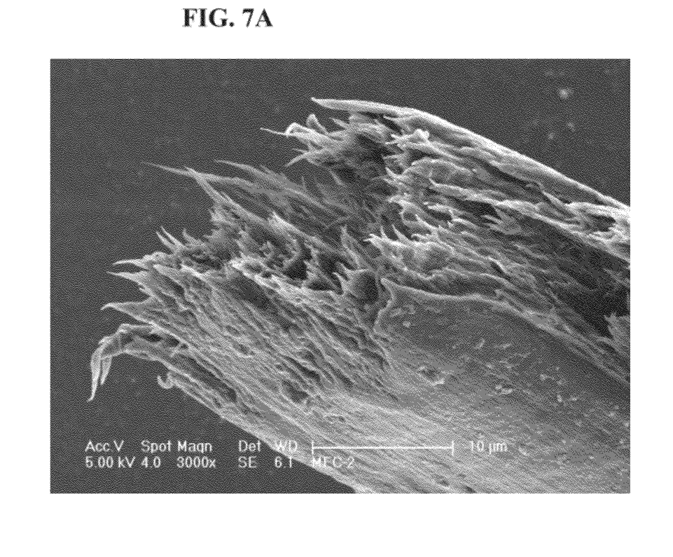

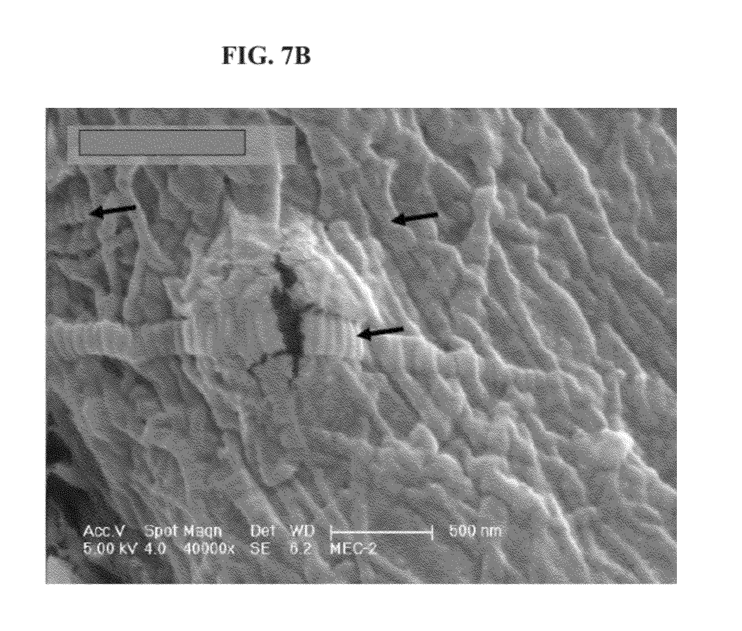

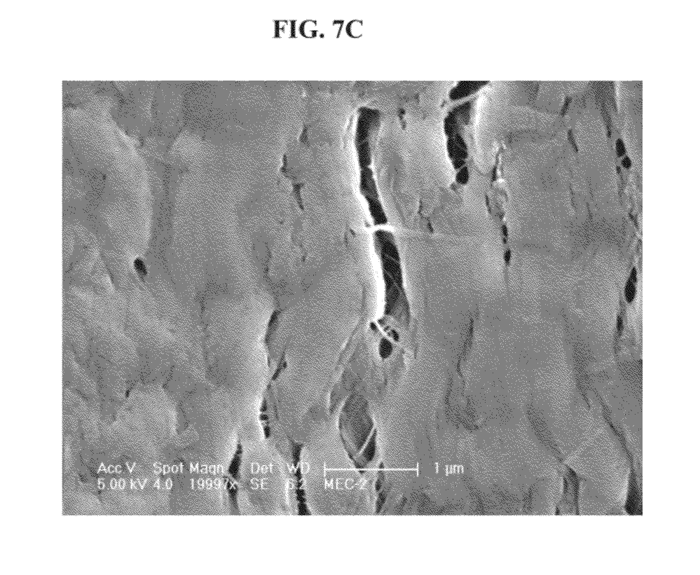

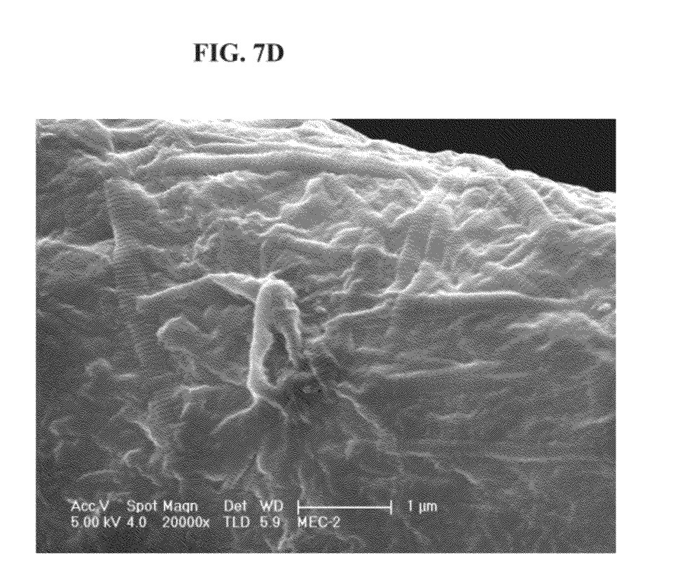

After extrusion into buffer 1 and 48 hours incubation in PBS, the fibers were partially swollen and were fragile. They were washed in water and dried in ethanol. The fiber surface clearly showed three different morphologies: an array of fibrils, between 250 to 50 nm in diameter, most of which were aligned with the fiber axis. Most of the fibrils displayed D banding with variable periodicity (FIG. 7B, arrows denote D banded areas of different periodicity). Other areas of the fiber displayed a cracked wood-like appearance, composed of single fibrils of ˜10-50 nm in diameter with areas of fused fibrils up to 1000 nm wide. The directionality of the fibers was with the fiber axis. D banded areas were sporadically observed (FIG. 7C, arrows denote D banded areas). Still other areas of the fibers displayed large fibrils, isotropic in their directionality with strong uniform D banding (FIG. 7D).

Buffer 2:

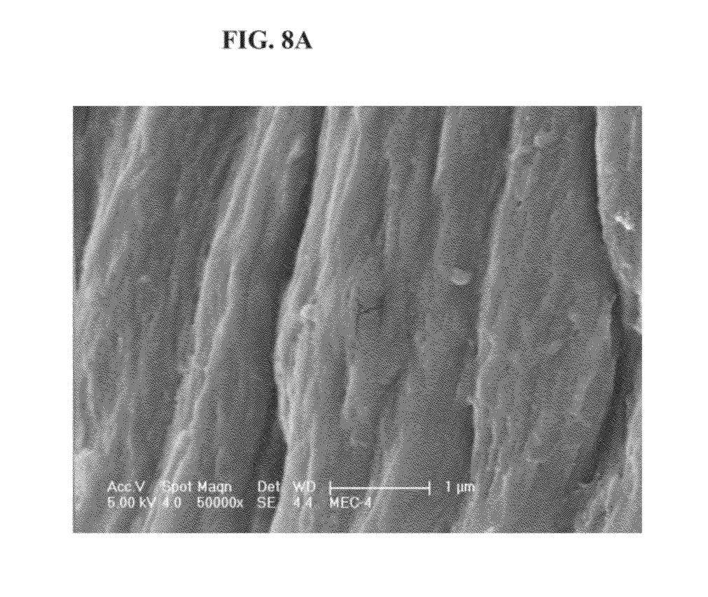

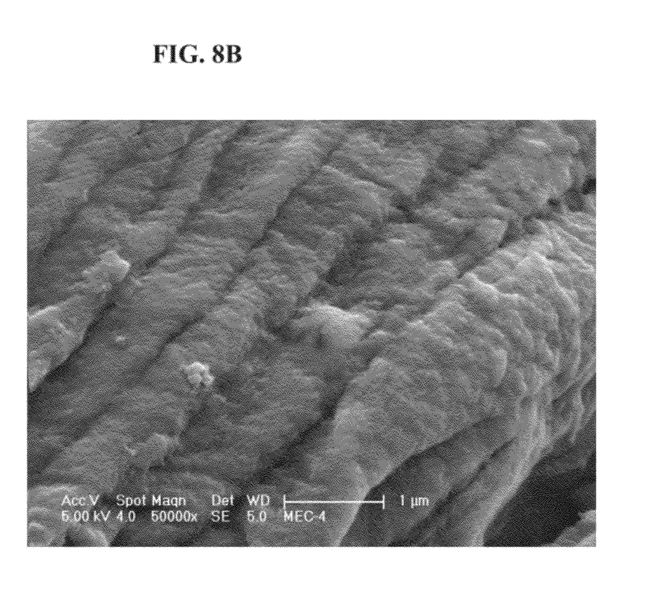

Fibers were injected into and incubated in buffer 2 for 48 hours at 34° C. Coagulation/incubation in high NaCl concentrations formed fibers that were composed of many small fibrils (˜10-20 nm), uniform in diameter (FIGS. 8A,B). The fiber surface appeared almost spongy, and the fibrils were tangled among themselves and are less aligned. No D banding was observed on the surface (FIGS. 8 A, B).

Buffer 2 and Subsequent Incubation in Buffer 3:

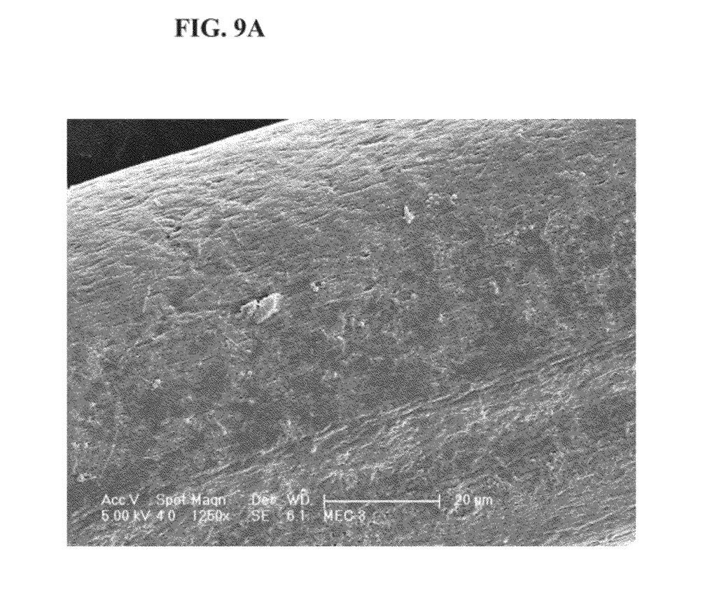

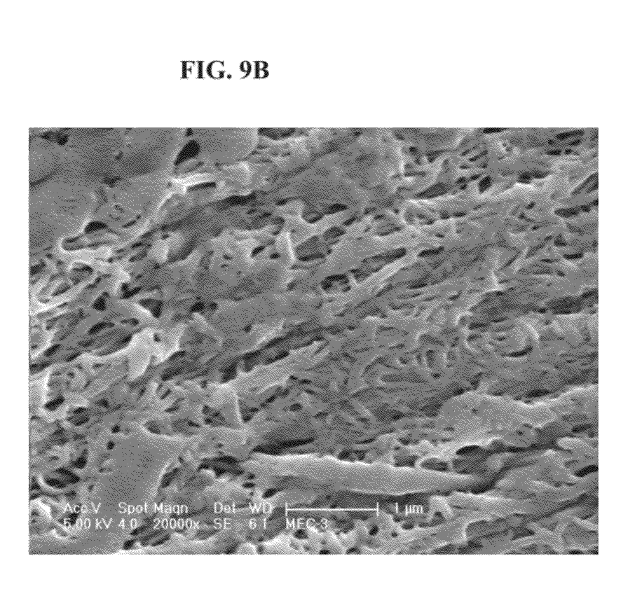

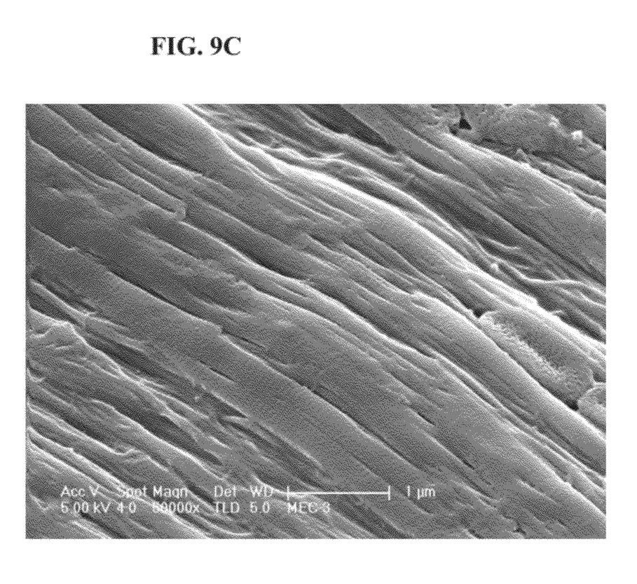

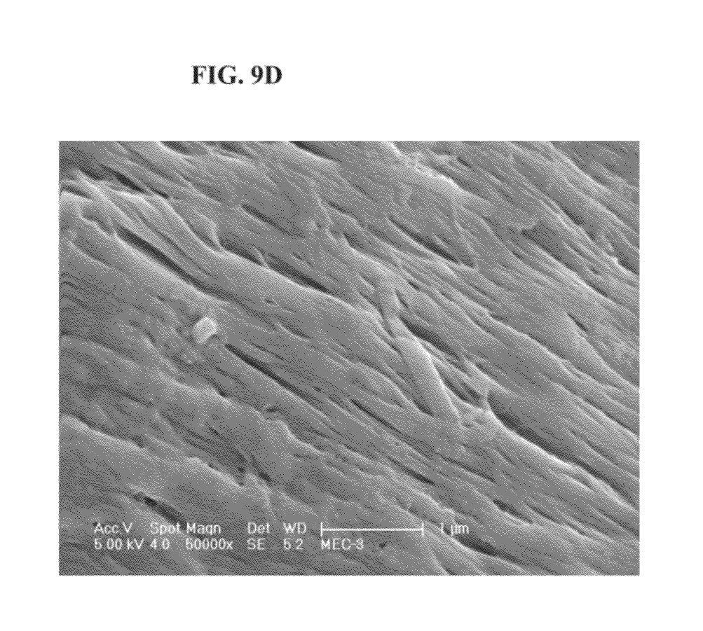

Fibers were injected into “buffer 2” and incubated therein for 20 minutes at 34° C. Subsequently, they were washed in water and transferred to “buffer 3” and incubated for 48 hours at 34° C. Some areas of the fibers displayed the same morphology of the fibers that were incubated in “buffer 2”—loosely aligned, tangled, small diameter fibrils. Other areas of the fiber showed an aligned array of D banded fibrils, very similar of natural tendon (FIGS. 9A-D).

Mechanical Studies of Fiber:

The results of the mechanical testing of the dried fibers revealed an s shaped curve as illustrated in Table 2, herein below.

TABLE 2

| | Stress at | | | |
|---|---|---|---|---|
| | break | Strain at | Force at | Modulus |
| Treatment | (MPa) | break % | break (N) | (GPa) |
| Ethanol as coagulation buffer | 153 | 29.2 | 0.300339 | 0.5 |
| Acetone as coagulation buffer | 211 | 18.3 | 0.135447 | 0.8 |
| Ispropanol as coagulation | 405 | 11.7 | 0.229671 | 2 |
| buffer | | | | |
| Fibrillogenesis Buffer | 239 | 17.3 | 0.284635 | 3 |
| followed by Ethanol Wash | | | | |

For comparison

| | | |
|---|---|---|
| Dry rat tail tendon | 366 | 0.14 |
| [Biomaterials 1989, Vol 10, | | |
| 38-42] | | |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and to individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of generating a collagen fiber, the method comprising extruding a solution of liquid crystalline collagen monomers into a coagulating solution, thereby generating the collagen fiber.

2. The method of claim 1, further comprising isolating the collagen fiber following said extruding.

3. The method of claim 2, further comprising drying the collagen fiber following said isolating.

4. (canceled)

5. The method of claim 3, further comprising polymerizing said collagen monomers following said extruding.

6. The method of claim 3, further comprising crosslinking said collagen monomers following said extruding.

7. The method of claim 1, wherein said extruding is effected using a spinneret.

8. The method of claim 1, wherein said coagulating solution further comprises a surfactant.

9. The method of claim 1, wherein said extruding is effected by passing through an orifice comprising an inner diameter of 30 ga.

10. The method of claim 1, wherein said coagulating solution comprises an organic solvent.

11. The method of claim 1, wherein said solution of crystalline collagen monomers comprises hyaluronic acid (HA).

12. The method of claim 1, wherein said solution of crystalline collagen monomers comprises a crosslinker.

13. The method of claim 10, wherein said at least one organic solvent is selected from the group consisting of acetone, ethanol and isopropanol.

14. The method of claim 1, wherein said collagen monomers comprise recombinant collagen monomers.

15. The method of claim 1, wherein said collagen monomers comprise animal-derived collagen monomers.

16. The method of claim 1, wherein said collagen monomers are present at a concentration of about 100 mg/ml in said solution of liquid crystalline collagen monomers.

17. The method of claim 1, wherein said liquid solution of crystalline collagen monomers is an acidic solution.

18. A collagen fiber produced by the method of claim 1.

19. The collagen fiber of claim 18, comprising an extinction pattern as displayed in FIGS. 1A-B.

20. A scaffold comprising the collagen fibers of claim 18.

21. A method of generating a collagen matrix, the method comprising:
(a) extruding a solution of liquid crystalline collagen monomers into a coagulating solution, thereby generating a collagen fiber; and
(b) casting the matrix from said collagen fiber, thereby generating the collagen matrix.
